(19) United States
(12) Patent Application Publication
(10) Pub. No.: US 2024/0368234 A1
(43) Pub. Date: Nov. 7, 2024

(54) SPYCATCHER ANALOGS AND USES THEREOF

(71) Applicant: BIO-RAD ABD SEROTEC GMBH, NEURIED (DE)

(72) Inventors: FRANCISCO YLERA, MUNICH (DE); MATEUSZ PUTYRSKI, NEURIED (DE); CHRISTIAN-MICHAEL ALOISIUS HEINZ HENTRICH, MUNICH (DE)

(21) Appl. No.: 18/655,747

(22) Filed: May 6, 2024

Related U.S. Application Data
(60) Provisional application No. 63/500,284, filed on May 5, 2023.

(51) Int. Cl. C07K 14/47; C07K 16/42; G01N 33/68

(52) U.S. Cl. CPC C07K 14/47; C07K 16/4258; G01N 33/68; C07K 2317/31; C07K 2319/00

(57) ABSTRACT

Lockable analogs of SpyCatcher, SnoopCatcher, DogCatcher, and SilkCatcher proteins, antigen binding proteins comprising lockable analogs of SpyCatcher proteins, nucleic acid constructs encoding the lockable analogs of SpyCatcher proteins are also provided. Vectors comprising the nucleic acid constructs, host cells comprising the vectors and nucleic acid constructs are, likewise, provided.

### CROSS-REFERENCE TO RELATED APPLICATION

This application claims the benefit of U.S. Provisional Application Ser. No. 63/500,284, filed May 5, 2023, the disclosure of which is hereby incorporated by reference in its entirety, including all figures, tables and amino acid or nucleic acid sequences.

### STATEMENT REGARDING SEQUENCE LISTING

The Sequence Listing for this application is labeled “Seq-List.xml” which was created on Apr. 30, 2024 and is 60,376 bytes. The entire content of the sequence listing is incorporated herein by reference in its entirety.

### BACKGROUND

The SpyTag/SpyCatcher system (Zakeri et al., 2012) and its improved versions SpyTag002/SpyCatcher002 (Keeble et al., 2017) and SpyTag003/SpyCatcher003 (Keeble et al., 2019) is a protein ligation technology that has been used for protein engineering in various fields and applications.

The SpyTag/SpyCatcher system is a widely used protein ligation technique. The present disclosure provides a new modified SpyCatcher which can be reversibly inhibited. In other words, the reactivity of SpyCatcher towards SpyTag can be turned on and off. This functionality provides control over when or where a SpyTagged protein can react with a SpyCatcher.

### BRIEF SUMMARY

The subject disclosure is directed to novel SpyCatcher analogs (also sometimes referred to as “a mutated SpyCatcher” or “mutated SpyCatchers”) comprising, for example, one or more novel cysteine mutation, such as a S59C mutation, in the SpyCatcher protein. While these mutations do not affect the catalytic activity of SpyCatcher towards SpyTag, the modification of the substituted amino acid residue(s) with, for example, molecules that form a covalent disulfide bond with the sulfur group of the cysteine abolishes or significantly reduces reactivity with SpyTag. This provides a means by which the catalytic activity of SpyCatcher can be controlled. For example, modification of the cysteine with disulfide-forming molecules such as Ellmann's reagent (5,5′-dithio-bis-(2-nitrobenzoic acid), DTNB) or HPDP-biotin (N-[6-(biotinamido)hexyl]-3′-(2′-pyridyldithio)propionamide) significantly reduces or abolishes the reactivity of SpyCatcher with SpyTag. SpyCatcher-SpyTag reactivity could be restored by treatment with reducing agents that cleave the disulfide bond (see FIG. 1). Similar mutations, such as the S59C mutation in SpyCatcher, can also be introduced into SpyCatcher002 and SpyCatcher003. Mutated SpyCatchers react with SpyTag and can be inhibited by disulfide bond formation as described above.

Introduction of cysteines into proteins is a common protein engineering step to enable site-specific conjugation via the introduced thiol group e.g. with maleimide chemistry. This has been shown for SpyCatcher, a protein without any endogenous cysteines, for a cysteine introduced at the N-terminus of the protein (Min et al., 2016). This has been applied to the SpyCatcher for S10C (Pessino et al., 2017) and S49C mutations (Dovala et al., 2016). The introduction of the cysteine into the SpyCatcher did not change its reactivity towards the SpyTag, even after conjugation of the cysteine to fluorescent dyes or DNA oligonucleotides. The difference of these mutations to the ones described here is that those thiol groups can be used for site-specific conjugation e.g. introduction of fluorescent dyes into the SpyCatcher without significantly changing its reactivity to SpyTag whereas the cysteine mutations introduced as described here inhibit the reactivity to SpyTag when modified by group covalently linked to the sulfur group of the cysteine residue.

In some embodiments, two amino acids can be exchanged to cysteines that can form an intra-chain disulfide bond under oxidizing conditions which then significantly reduces or abolishes the reactivity of SpyCatcher with SpyTag even without using Ellmann's reagent or other reagents that would form a disulfide bond with the sulfur atom of the cysteine residue. Upon cleavage of the disulfide bond with reducing reagents, the SpyCatcher reactivity with SpyTag can be restored.

Instead of the formation of a disulfide bond, photocleavable groups can be attached to the sulfur atom of one or more cysteine residues that are introduced into a SpyCatcher protein to form a SpyCatcher analog to which molecules, such as Ellmann's reagent or a photocleavable group, can be attached. As disclosed in Deng et al., various reagents (including photocleavable reagents) for modification of the sulfhydryl group of cysteine are known (see, for example, FIG. 1 which is hereby incorporated by reference in its entirety). These include photocleavable isoxazolinium derivatives, such as those disclosed in Deng et al. in FIG. 3 (each of which is hereby incorporated by reference in its entirety), which can be cleaved by irradiation with UV-A light (λₘₐₓ=365 nm) and, after treatment with NaBH₄, a native sulfhydryl group can be regenerated.

As an alternative to inserting one or more cysteines and reacting the thiol groups with a reagent (e.g., Ellmann's reagent), an artificial amino acid can be introduced at this position into the SpyCatcher. This artificial amino acid should contain a sufficiently bulky group to inhibit the SpyCatcher reactivity to SpyTag and the bulky group can be removed, e.g. by photocleavage or chemical uncaging, to restore the SpyCatcher-SpyTag reactivity. Yamaguchi App. Sci., 2022, 12:3750 (doi.org/10.3390/app12083750) discusses recent advances in protein caging tools, including a variety of amino acids that can be introduced into proteins (e.g., caged aspartic acid, caged serine, caged glycine, caged tyrosine, caged cysteine, and caged lysine caged phosphoserine and caged phosphotyrosine). Additional examples for such amino acids are listed in (Nodling et al., Essays in Biochemistry (2019) 63 237-266). Examples of unnatural amino acids consisting of photocaged cysteine include S—[(R,S)-1-{4′,5′-Dimethoxy-2′-nitrophenyl}ethyl]-L-cysteine (Ren W, Ji A, Ai H W. Light activation of protein splicing with a photocaged fast intein. J Am Chem Soc. 2015 Feb. 18; 137(6):2155-8. doi: 10.1021/ja508597d) and (2R)-2-Amino-3-{[({[1-(6-nitrobenzo[d][1,3]dioxol-5-yl)ethoxy]carbonyl}amino)methyl]thio}propanoic acid (Uprety R, Luo J, Liu J, Naro Y, Samanta S, Deiters A. Genetic encoding of caged cysteine and caged homocysteine in bacterial and mammalian cells. Chembiochem. 2014 Aug. 18; 15(12):1793-9. doi: 10.1002/cbic.201400073). Unnatural amino acids, which are not based on cysteine, can be used in a similar manner by selective uncaging of the unnatural amino acid. Such amino acids can be uncaged chemically or by light and can reversibly block the reactivity of SpyCatcher towards SpyTag. Examples of such unnatural amino acids include photocaged lysine, e.g., (S)-2-Amino-6-((((7-hydroxy-2-oxo-2H-chromen-4-yl)methoxy)carbonyl)amino)hexanoic acid (Luo J, Uprety R, Naro Y, Chou C, Nguyen D P, Chin J W, Deiters A. Genetically encoded optochemical probes for simultaneous fluorescence reporting and light activation of protein function with two-photon excitation. J Am Chem Soc. 2014 Nov. 5; 136(44):15551-8. doi: 10.1021/ja5055862), photocaged tyrosine, e.g., (2S)-2-Amino-3-(4-(1-(6-nitrobenzo[d][1,3]dioxol-5-yl)ethoxy)phenyl)propanoic acid (Luo J, Torres-Kolbus J, Liu J, Deiters A. Genetic Encoding of Photocaged Tyrosines with Improved Light-Activation Properties for the Optical Control of Protease Function. Chembiochem. 2017 Jul. 18; 18(14):1442-1447. doi: 10.1002/cbic.201700147) or chemically caged lysine, e.g., N6-(((4-azidobenzyl)oxy)carbonyl)-L-lysine (Ge Y, Fan X, Chen P R. A genetically encoded multifunctional unnatural amino acid for versatile protein manipulations in living cells. Chem Sci. 2016 Dec. 1; 7(12):7055-7060. doi: 10.1039/c6sc02615j). Additionally, amino acids with selectively removable bulky substituents can be introduced to SpyCatcher and other proteins of interest by peptide synthesis. An example of such an amino acid is cysteine caged with 6-bromo-7-hydroxy-3-methylcoumarin-4-ylmethyl (Mahmoodi et al., 2016, Org. Biomol. Chem., 14:8289). This cage can be removed by photoirradiation (e.g., with one or two photon irradiation). Each of these references is hereby incorporated by reference in their entireties, particularly with respect to the unnatural caged amino acids disclosed therein.

The subject disclosure provides for different applications for the disclosed SpyCatcher analogs. With immobilized mutated SpyCatchers that are locked with an appropriate disulfide forming molecule, SpyTagged proteins can be captured onto an array at specific position by unlocking only certain SpyCatchers. This provides a means by which complicated patterns of different SpyTagged proteins could be immobilized on an array. For example, photocleavable reagents can be used for such an application (Deng et al., 2019, Communications Chemistry, 2:93, which is hereby incorporated by reference in its entirety).

Bispecific antibodies are an important class of drugs, particularly in oncology. Many technologies exist to generate bispecific antibodies in their final format (Ma et al., 2021). There is however an unmet need in the discovery of bispecific antibodies: there is no simple and efficient way to generate prototype bispecific antibodies during drug development. In most cases, both halves of the antibody must be cloned into different expression vectors with different modifications (e.g., Antibody1-SpyTag, Antibody2-SpyCatcher, Knob-in-holes IgGs etc.). In other cases, the efficiency of bivalent antibody generation is poor (intein-based approaches, AlphaThera+SpyTag/SpyCatcher). The disclosed mutated SpyCatchers can be used for the rapid generation of bispecific antibodies for screening of large numbers of different antibody sets for the best combination of candidates. For example, it is possible to generate a SpyCatcher003 dimer (BiCatcher003) with a regular SpyCatcher003 fused to a S59C mutated SpyCatcher003. After locking the mutated cysteine with a disulfide-forming reagent and coupling the first SpyTagged antibody to the regular SpyCatcher, the disulfide bond of the mutated Catcher is cleaved and the second SpyTagged antibody is added for coupling (FIG. 2). The advantage of this method over many other methods for the generation of bispecific antibodies is the use of the same antibody format for the first and second antibody. This avoids additional subcloning, expression, and purification steps and dramatically accelerates the process: generation of bispecifics in high throughput can easily be done within a few hours.

The use of HPDP-biotin as a reversible protection group additionally offers a quick and convenient way of purifying the Antibody1-SpyCatcher-locked SpyCatcher intermediate after coupling with the first antibody: it can be adsorbed to a solid phase coated with avidin (e.g., Streptavidin beads or Streptavidin Agarose, Neutravidin etc.) or Streptactin resin/beads, washed, and simultaneously unlocked and released with a reducing agent. By removing any unconjugated first antibody this way, a higher yield of bispecific product can be generated. Alternatively, 3-nitro-2-pyridylsulfenyl (Npys) activated affinity tag peptides, e.g., Npys-Cys-StrepTag2 in combination with a Streptactin resin purification step, can be used in a similar fashion. Other purification tags such as FLAG-, myc-, HA-, V5-, His-tag and others activated by Npys (e.g., Npys-Cys-His6) can be used in combination with the respectively matching purification matrix. The purified product can then be coupled to the second antibody without interference of the first antibody.

The first product resulting from phage display antibody selection is usually an antibody fragment that either carries a SpyTag, when using SpyDisplay (Kellmann et al, 2023 which is hereby incorporated by reference in its entirety), or is an antibody fragment, e.g., a Fab, which can be subcloned into an expression vector that fuses a SpyTag sequence to the C-terminus of the antibody fragment, in case other display methods were applied. After a simple bacterial production, these SpyTagged Fabs are ready for a high throughput generation of bispecific antibodies.

Thus, in one aspect of the invention, SpyCatcher analogs comprising one or more cysteine mutations, such as a S59C mutation, in the SpyCatcher protein are provided. As discussed above, these mutations do not affect the catalytic activity of SpyCatcher towards SpyTag; however, the modification of this amino acid residue with e.g., a molecule that abolishes or significantly reduces reactivity with SpyTag. SpyCatcher analogs may be referred to as “SpyLock” proteins when the cysteine group that has been introduced into the SpyCatcher analog is blocked with a disulfide bond forming reagent such as Ellmann's reagent (5,5′-dithio-bis-(2-nitrobenzoic acid), DTNB), HPDP-biotin (N-[6-(biotinamido)hexyl]-3′-(2′-pyridyldithio)propionamide) or a 3-nitro-2-pyridylsulfenyl (NPys) activated cysteine-containing peptide (e.g., affinity tag). Alternatively, a reagent with a photocleavable linker such as 6-bromo-7-hydroxy-3-methylcoumarin-4-ylmethyl (mBhc) or a photocleavable isoxazolinium compound that can specifically react with the thiol group of the cysteine.

In another aspect of the invention, SpyCatcher analogs comprising one or more unnatural amino acid with a bulky group which abolishes or significantly reduces reactivity with SpyTag. This bulky group can be removed by photocleavage or chemical uncaging to restore the SpyCatcher-SpyTag reactivity. Examples for such unnatural amino acids include S—[(R,S)-1-{4′,5′-Dimethoxy-2′-nitrophenyl}ethyl]-L-cysteine, (2R)-2-Amino-3-{[({[1-(6-nitrobenzo[d][1,3]dioxol-5-yl)ethoxy]carbonyl}amino)methyl]thio}propanoic acid, (S)-2-Amino-6-((((7-hydroxy-2-oxo-2H-chromen-4-yl)methoxy)carbonyl)amino)hexanoic acid, (2S)-2-Amino-3-(4-(1-(6-nitrobenzo[d][1,3]dioxol-5-yl)ethoxy)phenyl)propanoic acid, N6-(((4-azidobenzyl)oxy)carbonyl)-L-lysine or a cysteine caged with 6-bromo-7-hydroxy-3-methylcoumarin-4-ylmethyl.

In another aspect of the invention, fusion proteins comprising one or more of the disclosed SpyCatcher analogs (multimeric fusion proteins which may also be referred to as BiCatchers or MultiCatchers), optionally joined by a linker, are provided. In some embodiments, the multimeric fusion proteins comprise a plurality of SpyCatcher analogs (also referred to as a MultiCatcher) or a pair of SpyCatcher proteins, one of which is a SpyCatcher analog (a BiCatcher).

Thus, the multimeric fusion proteins can comprise one or more SpyCatcher analog linked to one or more non-mutated SpyCatcher proteins. The fusion proteins can be depicted in the format (A-B)ₙ, (B-A)ₙ, (A-L-B)ₙ, (B-L-A)ₙ, Aₘ-Bₒ, Bₘ-Aₒ, Aₘ-L-Bₒ, or Bₘ-L-Aₒ, where A is a SpyCatcher analog, L is a linker, B is a non-mutated SpyCatcher protein, and n is >1, m is >1, and o is >1. When n=1, the fusion proteins are referred to as BiCatchers and when n is greater than 1, the fusion proteins are referred to as MultiCatchers and additional elements of the fusion protein can be added to the MultiCatcher directly or via peptide linkers. When m and o equal 1, the fusion proteins are referred to as BiCatchers and when either, or both, m and o are greater than 1, the fusion proteins are referred to as MultiCatchers and additional elements of the fusion protein can be added to the MultiCatcher directly or via peptide linkers. As would be apparent to those skilled in the art, a variety of multimeric fusion proteins can be provided in which at least one of the SpyCatcher proteins is a SpyCatcher analog and the other SpyCatcher proteins of the multimeric fusion protein are non-mutated SpyCatcher proteins. In such embodiments, the discrete elements of the multimeric fusion protein (i.e., the SpyCatcher analog and non-mutated SpyCatcher proteins) can be randomly ordered and can be, optionally, connected by a linker. Non-limiting examples of such fusion proteins are provided in SEQ ID NOs: 5 and 6. In these examples, the mutated SpyCatcher is the N-terminal protein in SEQ ID NO: 5 and the C-terminal protein in SEQ ID NO: 6.

The disclosed SpyCatcher analogs and fusion proteins can further comprise a tag. For example, a tag such as His-tag can also be added at the N- or C-terminus of the disclosed SpyCatcher analogs or a multimeric fusion protein to facilitate purification of the fusion protein by affinity chromatography. A protease cleavage site such as the TEV protease site can also be added between the tag (e.g., a His-tag) and the SpyCatcher motifs of the fusion protein to permit removal of the tag after affinity chromatography. As discussed above, a “locked” SpyCatcher is locked with a reagent blocking the free sulfhydryl group of the introduced cysteine, such as a photocleavable protecting groups or chemically cleavable groups such as Ellmann's reagent, 3-nitro-2-pyridylsulfenyl (Npys) or HPDP-biotin. As discussed above, a “locked SpyCatcher” can be purified using avidin chromatography, avidinated beads or Streptactin resin or beads (for HPDP-biotin locked SpyCatcher), with a Streptactin resin or beads purification step in the case of a Npys-Cys-StrepTag2 locked SpyCatcher or other Npys-activated peptide tags with their matching resin.

Other aspects of the invention provide antigen binding proteins, nucleic acid constructs encoding the antigen binding proteins, vectors comprising the nucleic acid constructs, and host cells comprising the vectors and nucleic acid constructs. Kits comprising components to make the antigen binding proteins are also provided.

In an embodiment, the antigen binding protein comprises two or more antigen binding fragments, each comprising a first binding motif (e.g., a SpyTag, SpyTag002, or SpyTag003), and a fusion protein comprising two or more second binding motifs, at least one of which is a SpyLock are provided. The first binding motifs joined to the antigen binding fragments and the two or more second binding motifs of the fusion protein (at least one of which a SpyLock protein) may, optionally, be joined by one or more linker sequence. The first binding motifs of the two or more antigen binding fragments are covalently conjugated to the fusion protein comprising two or more second binding motifs via protein ligation. In some embodiments, the fusion protein is a dimer or multimer of second binding motifs, at least one of which is a SpyLock protein which are, optionally, joined by a linker sequence. In some embodiments, the antigen binding protein may further comprise a detectable label (e.g., a fluorophore, a fluorescent protein, biotin, or an enzyme). In some embodiments, the linker sequence is 1-5 amino acids long. In some embodiments, the linker sequence is GGGGS (SEQ ID NO: 60).

In a similar way, this method to identify reversibly inhibitable SpyCatcher analogs can be transferred to other protein ligation pairs such as SnoopTag/SnoopCatcher (Veggiani et al. 2016), DogTag/DogCatcher (Keeble et al. 2022), SilkTag/SilkCatcher (Fan et al. 2023).

Also provided are nucleic acid constructs encoding the antigen binding proteins and vectors comprising the nucleic acid constructs. Also provided are host cells comprising the nucleic acid constructs or vectors. Also provided are kits comprising components for making the antigen binding proteins.

### BRIEF DESCRIPTION OF THE DRAWINGS

FIGS. 1A-1C. 1A) Mutated S59C SpyCatcher reacts with Spytagged Fab (or protein) to form the SpyTag/SpyCatcher ligation product. 1B) After inhibition through disulfide bond formation with e.g. HPDP-biotin the mutated SpyCatcher does not react with SpyTag anymore. 1C). After disulfide bond cleavage with a reducing agent, reactivity of the SpyCatcher to SpyTag is restored.

FIG. 2 illustrates generation of bispecific antibodies with an SpyCatcher-mutated SpyCatcher fusion protein.

FIG. 3. Time course (SDS PAGE) of the reaction of HPDP-biotin modified SpyCatcher S59C with and without TCEP.

FIG. 4. Time course (SDS PAGE) of the reaction of HPDP-biotin modified SpyCatcher002 S59C with and without TCEP.

FIG. 5. Time course (SDS PAGE) of the reaction of HPDP-biotin modified SpyCatcher003 S59C with and without TCEP.

FIG. 6. Coupling kinetic of SpyCatcher003 S59C-SpyCatcher003 (left) and SpyCatcher003-SpyCatcher003 S59C (right) after labeling with HPDP-biotin.

FIG. 7. Unlocking of SpyLock with reducing agents, 1 h at room temperature. SDS PAGE.

FIG. 8. Generation of 4 bispecific antibodies using SpyLock (SL). Input Fabs (left) and coupled bispecific antibodies (right). SDS-PAGE. Ocre: anti-Ocrelizumab Fabs. Dupi: anti-Duplilumab Fabs.

FIGS. 9A-9B. Sandwich ELISAs with the bispecific anti-idiotypic antibody constructs. FIG. 9A—Ocrelizumab coated, detection with Dupilumab-HRP. FIG. 9B—Dupilumab coated, detection with Ocrelizumab-HRP.

FIG. 10. Sandwich ELISA of bispecific anti-idiotypic antibodies against Dupilumab and Ocrelizumab measured in both orientations in comparison with monospecific controls generated under identical conditions.

FIG. 11. Constructs produced by cysteine scanning mutagenesis.

FIGS. 12A-12B. Coupling of rationally designed SpyLock-candidates to MBP. FIG. 12A shows QC of unlabeled Catchers. FIG. 12B shows coupling of HPDP-biotin modified Catchers for 1 hour in presence of varying concentrations of reducing agent.

FIG. 13. Coupling ability of the Cys-Catchers resulting from Cys scanning. Bottom right is a table translating lane number to position of the amino acid mutated to cysteine. The two controls which are supposed to be unreactive are in Lanes 9 and 52. 1 h coupling time.

FIG. 14. Coupling of DTNB-modified SpyCatchers. Each gel features a pair of input catcher and coupling product with MBP-SpyTag. Control is the SpyCatcher002 version of SpyLock S59C. Lane number as in FIG. 13. 1.5 h coupling time.

FIG. 15. Coupling of DTNB-modified SpyCatchers with MBP-SpyTag in absence or presence of reducing agent (10 mM TCEP, 1.5 h coupling time).

FIG. 16. Fab stability after extended exposure to TCEP. Fab fragments were incubated with or without 5 mM TCEP for 16 hours. This exposure time did not change their affinity as measured by BLI.

FIG. 17. Fab stability after extended exposure (one week) to TCEP. Fab fragments were incubated with or without 5 mM TCEP for an entire week. GFP was coated, anti-GFP Fabs were titrated and detection was performed with anti-Fab-HRP. This length of exposure resulted in only minor effects on antigen binding ability as measured by titration ELISA.

### DETAILED DESCRIPTION

SpyCatcher analogs, SpyLock proteins, antigen binding proteins comprising at least one SpyCatcher analog, nucleic acid constructs encoding the antigen binding proteins and/or SpyCatcher analogs, vectors comprising the nucleic acid constructs, host cells comprising the vectors and nucleic acid constructs, and kits for making antigen binding proteins that comprise at least one SpyCatcher analog or a SpyLock protein are provided. The antigen binding proteins are covalently linked to monomeric SpyCatcher analogs or to multimeric fusion proteins comprising at least one SpyCatcher analog to create either monospecific (recognize and bind to one antigen) or bispecific (i.e., bind to two different antigens or two different epitopes on the same antigen) constructs. Multimeric antigen binding proteins (comprising three or more antigen binding proteins) can be formed by using a MultiCatcher comprising at least one of the disclosed SpyCatcher analogs. The multimeric antigen binding proteins can be monospecific or specific for two or more antigens. For example, a trimeric antigen binding protein can bind to three different antigens or to two different antigens in an instance where two antigen binding fragments bind to the same antigen and the third antigen binding fragment binds to a different antigen). As another example, a multispecific tetrameric antigen binding protein can bind to two, three, or four different antigens depending on the specificities of the antigen binding fragments used to construct the multimeric antigen binding protein.

The multimeric antigen binding proteins either have higher valency than monomers, contain additional functions, or are bispecific, or are a combination thereof. The antigen binding proteins are made by protein ligation which circumvents the genetic engineering steps currently needed to make such binding reagents. Multivalency increases the sensitivity of the antigen binding proteins which is a useful characteristic in such applications as Western blotting, flow cytometry, immunohistochemistry, in some enzyme-linked immunoassays, and for therapeutic use. Bispecificity or multispecificity is useful for increasing target specificity, target affinity or for binding to two or more different antigens simultaneously. Bispecific antigen binding proteins are also of increasing significance as therapeutic drugs. Addition of a second function by protein ligation is useful for preparing labeled antigen binding proteins for applications such as Western blotting, flow cytometry, immunohistochemistry, enzyme-linked immunoassays, for directed immobilization, or for other applications where a second function is required, such as creation of antibody-enzyme fusion proteins for cancer therapy.

### Definitions

Unless otherwise stated, the following terms used in this application, including the specification and claims, have the definitions given below.

As used herein, the singular forms “a”, “an” and “the” are intended to include the plural forms as well, unless the context clearly indicates otherwise. Furthermore, to the extent that the terms “containing”, “including”, “includes”, “having”, “has”, “with”, or grammatical variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “and/or” as used herein refers to and encompasses any and all possible combinations of one or more of the associated listed items. For example, the phrase “A, B, and/or C” includes A alone, B alone, C alone, the combination of A and B, the combination of A and C, the combination of B and C, and the combination of A, B, and C. Also, the term “or” is used in its inclusive sense (and not in its exclusive sense) so that when used, for example, to connect a list of items, the term “or” means one, some, or all of the items in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z).

The phrases “consisting essentially of” or “consists essentially of” indicate that the claim encompasses embodiments containing the specified materials or steps and those that do not materially affect the basic and novel characteristic(s) of the claim.

The term “about” or “approximately” means within an acceptable error range for the particular value as determined by one of ordinary skill in the art, which will depend in part on how the value is measured or determined, i.e., the limitations of the measurement system. The terms “about” and “approximately” are meant to encompass a range of 20%, ±10% or ±5% of a given value. Thus, in the context of compositions containing amounts of ingredients where the terms “about” or “approximately” are used, these compositions can contain the stated amount of the ingredient with a variation (error range) of 0-10% around the value (X±10%). In the context of pH, the term “about” or “approximately” encompasses a range of ±0.2 units of a given value.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

The terms “disulfide forming reagent”, “locking agent”, “disulfide-forming molecule”, or “disulfide-forming molecules” is used to define a reagent that can form a disulfide bond with a thiol group of a cysteine that has been introduced into the SpyCatcher analog in an aqueous buffered solution which is generally tolerated by most proteins. In certain embodiments, the formation of the disulfide bond is complete or nearly complete (at least 80% complete or higher) within about 1 to about 24 hours. These terms can be used interchangeably within this disclosure. Non-limiting examples of disulfide forming reagents include: methyl methanethiosulfonate and other methanethiosulfonates, 2,2′-dipyridyl disulfide and derivatives thereof, 5,5′-dithiobis-(2-nitrobenzoic acid) and derivatives thereof, oxidized glutathione (GSSH) and derivatives thereof, cystamine and derivatives thereof, cystine and derivatives thereof, pyridyldithiol-Biotin and other pyridyldithiols/pyridyldithio containing small molecules/chemicals, and 1,2,4-thiadiazoles and their derivatives and any type of reagent that can form disulfide bonds within an aqueous solution can be used.

The term “SpyCatcher analog”, as used herein, refers to SpyCatcher proteins into which mutations have been introduced that do not affect the catalytic activity of SpyCatcher towards SpyTag but which permit for the modification of SpyCatcher with, for example, molecules, such that the modification abolishes or significantly reduces reactivity with SpyTag. This provides a means by which the catalytic activity of SpyCatcher can be controlled. The mutations can comprise the introduction of a cysteine residue and/or an unnatural amino acid as discussed below.

A “mutated SpyCatcher analog” refers to a SpyCatcher analog that contains additional mutations within the SpyCatcher analog. Mutated SpyCatcher analogs have at least 60%, 65%, 70%, 75%, 80%, 85%, 90%, 91%, 92%, 93%, 94%, 95%, 96%, 97%, 98%, or 99% sequence identity to a given SpyCatcher analog, with the proviso that said mutated SpyCatcher analog has at least 60% sequence identity to a particular SpyCatcher analog and the mutated SpyCatcher analog catalyzes protein ligation (the isopeptide bond forming Lys31 and the catalytic Glu77 are not mutated/substituted) and the mutation(s) initially introduced into the SpyCatcher analog (the one or more cysteine residue and/or unnatural amino acid as disclosed herein) is/are not substituted in said mutated SpyCatcher analog having at least 60% sequence identity.

The terms “SpyCatcher” or “SpyCatcher protein” may, in certain contexts, refer to any of SpyCatcher (SEQ ID NO: 47), SpyCatcher short (SEQ ID NO: 50), SpyCatcher002 (SEQ ID NO: 48), or SpyCatcher003 (SEQ ID NO: 49). When used generically, the terms will be clear from the context of this disclosure and refer to non-mutated SpyCatcher proteins.

“Antibody” refers to an immunoglobulin, composite (e.g., fusion), or fragmentary form thereof. The term includes but is not limited to polyclonal or monoclonal antibodies of the classes IgA, IgD, IgE, IgG, and IgM, derived from antibody-producing cell lines or from in vitro antibody libraries, including natural or genetically modified or synthetic forms such as humanized, human, single-chain, chimeric, synthetic, recombinant, hybrid, mutated, grafted, and other in vitro generated antibodies. “Antibody” also includes composite forms including but not limited to fusion proteins having an immunoglobulin moiety.

As used herein, the phrase “antigen binding fragments” refers to protein structures comprising antigen-binding capabilities. They comprise polypeptides derived from antibodies and affinity ligands obtained from other structures. Examples for antibody-derived fragments are Fab fragments, the variable fragment (Fv), the disulfide-stabilized variable fragment (dsFv), the single-chain variable fragment (scFv), the diabody or the single-chain Fab fragment (scFab). Further examples include forms of antibody fragments that contain a single-domain of the antigen binding site including variable domain of heavy chain antibodies (VHH), single-domain antibodies (sdAbs), or variable New Antigen Receptors (vNAR) from cartilaginous fish. Furthermore, non-antibody scaffolds with antigen-binding capabilities such as Variable Lymphocyte Receptors (VLRs), affimers (derived from the cysteine protease inhibitor family of cystatins), affibodies (based on the Z domain of the IgG-binding staphylococcal protein A), darpins (designed ankyrin repeat proteins, which use a scaffold where a varying number of structural motifs or repeats are stacked to form the repeat protein domain), ArmRPs (Armadillo repeat proteins is a modular peptide-binding scaffold) anticalins (derived from the diverse set of lipocalin proteins), monobodies (that use the 10th fibronectin type III domain of human fibronectin as a scaffold), Adnectins, microbodies, Kunitz domains, Affilins, Tetranectins, Avimers, or any other antigen-binding peptide or polypeptide are included in the definition.

One or more linker sequences (e.g., a glycine/serine rich linker) may flank the binding motifs to enhance accessibility for reaction or to enhance flexibility of the fused polypeptides. In some embodiments, one or more linker sequences flank both the N- and C-terminus of a binding motif to enhance accessibility for reaction or to enhance flexibility of the fused polypeptides (e.g., in the case of a fusion protein comprising multimeric binding motifs). The phrase “joined by a linker sequence”, as used herein, in permits the use of one or more linker sequences to connect two or more binding motifs, a binding motif and a polypeptide or a binding motif and an antigen binding fragment. Where a plurality of linker sequences are used to join binding motifs, to join antigen binding fragments to a binding motif, or to join a polypeptide and a binding motif, the linker sequences can be the same or different.

The term “prokaryotic system” refers to prokaryotic cells such as bacterial cells or prokaryotic phages or bacterial spores. The term “eukaryotic system” refers to eukaryotic cells including cells of animal, plants, fungi and protists, and eukaryotic viruses such as retrovirus, adenovirus, baculovirus. Prokaryotic and eukaryotic systems may be, collectively, referred to as “expression systems”.

As used herein the term “vector” refers to a nucleic acid molecule, preferably self-replicating within a cell, which transfers an inserted nucleic acid molecule into and/or between host cells. Typically vectors are circular DNA comprising a replication origin, a selection marker, and/or viral package signal, and other regulatory elements. Vector, vector DNA, plasmid DNA, phagemid DNA are interchangeable terms in description of this invention. The term includes vectors that function primarily for insertion of DNA or RNA into a cell, replication vectors that function primarily for the replication of DNA or RNA, and expression vectors that function for transcription and/or translation of the DNA or RNA. Also included are vectors that provide more than one of the above functions.

As used herein the term “expression vector” is a polynucleotide which, when introduced into an appropriate host cell, leads under appropriate conditions to the transcription and translation of one or more polypeptides. The term “expression vector”, refers to vectors that direct the expression of polypeptides of interest fused in frame with a binding motif.

In the context of this application, the term “binding motif” refers to one of the elements needed for protein ligation, such as SpyTag, SpyTag002, SpyTag003, SnoopTag, DogTag, SilkTag, SpyCatcher, SpyCatcher002, SpyCatcher003, SnoopCatcher, DogCatcher, and SilkCatcher. These elements may be referred to as a “first binding motif” and a “second binding motif’, it being understood that the first and second binding motifs are able to undergo protein ligation either spontaneously or with the help of an enzyme (e.g., SpyTag with SpyCatcher).

The binding motifs used in a BiCatcher or MultiCatcher can be orthogonal. As used herein, the term “orthogonal” refers to mutually unreactive or non-cognate binding motif pairs (e.g., SpyTag and SpyCatcher cannot react with either of SnoopCatcher or SnoopTag to form an isopeptide bond).

The term “solid support” (and grammatical equivalents of these terms) are used to denote a solid inert surface or body to which an agent, such as an antibody or a peptide (e.g., SpyTag, SpyTag002, SpyTag003, SnoopTag, DogTag, and SilkTag), or protein (e.g., SpyCatcher or a SpyCatcher analog as disclosed herein) can be immobilized or affixed. These terms (“solid support” (and grammatical equivalents of these terms)) may be used interchangeably. Non-limiting examples of a solid support include plastic, polystyrenes, nitrocellulose, membranes, chips, plates, and particles. A solid support can be a particle or a planar support such as glass, polymeric or silica chips (such as microchips), plates (e.g., microtiter plates), slides, etc. The agent can be immobilized on the surface of the solid support at specific locations (e.g., in specific wells of a plate (e.g., microtiter plate) or at specific locations on a chip, microchip, plate or slide).

As used herein the terms “polynucleotides”, “nucleic acids” and “oligonucleotides” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the nucleotide polymer.

As used herein the term “amino acid” refers to either natural and/or unnatural or synthetic amino acids, both the D or L optical isomers, amino acid analogs.

As used herein the terms “polypeptide”, “peptide”, and “protein,” are used interchangeably herein to refer to polymers of amino acids of any length and peptidomimetics.

As used herein the term “host cell” includes an individual cell or cell culture which can be, or has been, a recipient for the disclosed expression constructs. Host cells include progeny of a single host cell. The progeny may not necessarily be completely identical to the original parent cell due to natural, accidental, or deliberate mutation.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

Sequences are “substantially identical” to each other if they have a specified percentage of nucleotides or amino acid residues that are the same (e.g., at least 60%, 65%, 70%, 75%, 80%, 85%, 90%, 91%, 92%, 93%, 94%, 95%, 96%, 97%, 98%, or 99% identity over a specified region or the entire designated sequence if a region is not specified), when compared and aligned for maximum correspondence over a comparison window.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, about 10 to about 300, about 10 to about 150 in which a sequence may be compared to a reference sequence of the same number of contiguous positions after the two sequences are optimally aligned. The comparison window can also be the entire length of either the reference or the test sequence.

Percent sequence identity and sequence similarity can be determined using the BLAST 2.0 algorithm, which is described in Altschul et al. (J. Mol. Biol. 215:403-10, 1990). Software for performing BLAST 2.0 analyses is publicly available through the National Center for Biotechnology Information (see Worldwide Website: ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915 (1989)) alignments (B) of 50, expectation I of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The terms “label” or “detectable label” refer to a composition detectable by spectroscopic, photochemical, biochemical, immunochemical, chemical, or other physical means. For example, useful labels include fluorescent dyes (fluorophores), fluorescent quenchers, luminescent agents, electron-dense reagents, enzymes (e.g., as commonly used in an ELISA), biotin, digoxigenin, ³²P and other isotopes, haptens, proteins, nucleic acids, or other substances which can be made detectable, e.g., by incorporating a label into an oligonucleotide or peptide. Other non-limiting examples include split proteins including, but not limited to: self-associating split fluorescent proteins (SAsFPs), for example: split GFP_{1-10/11}, sSplit sfCherry2_{1-10/11}, split sfCherry3_{1-10/11} and self-associating split luciferases, for example split nanoLuc luciferase. The term includes combinations of single labeling agents, e.g., a combination of fluorophores that provides a unique detectable signature, e.g., at a particular wavelength or combination of wavelengths.

### Modified Spycatcher Proteins (SpyCatcher Analogs)

As discussed above, the subject disclosure provides SpyCatcher analogs that retain the ability to form an isopeptide bond with SpyTag. The SpyCatcher analogs are:

SEQ ID NO: 1: SpyCatcher S59C
VDTLSGLSSEQGQSGDMTIEEDSATHIKFSKRDEDGKELAGATMELRDSSGKTISTWI
CDGQVKDFYLYPGKYTFVETAAPDGYEVATAITFTVNEQGQVTVNGKATKGDAHI
SEQ ID NO: 2: SpyCatcher002 S59C
VTTLSGLSGEQGPSGDMTTEEDSATHIKFSKRDEDGRELAGATMELRDSSGKTISTWI
CDGHVKDFYLYPGKYTFVETAAPDGYEVATAITFTVNEQGQVTVNGEATKGDAHT
SEQ ID NO: 3: SpyCatcher003 S59C
VTTLSGLSGEQGPSGDMTTEEDSATHIKFSKRDEDGRELAGATMELRDSSGKTISTWI
CDGHVKDFYLYPGKYTFVETAAPDGYEVATPIEFTVNEDGQVTVDGEATEGDAHT
SEQ ID NO: 4: SpyCatcher short S38C
DSATHIKFSKRDEDGKELAGATMELRDSSGKTISTWICDGQVKDFYLYPGKYTFVET
AAPDGYEVATAITFTVNEQGQVTVNG
SEQ ID NO: 5: SpyCatcher003 S59C-SpyCatcher 003
VTTLSGLSGEQGPSGDMTTEEDSATHIKFSKRDEDGRELAGATMELRDSSGKTISTWI
CDGHVKDFYLYPGKYTFVETAAPDGYEVATPIEFTVNEDGQVTVDGEATEGDAHTG
SSGSGGGGSGGGGSGVTTLSGLSGEQGPSGDMTTEEDSATHIKFSKRDEDGRELAGA
TMELRDSSGKTISTWISDGHVKDFYLYPGKYTFVETAAPDGYEVATPIEFTVNEDGQ
VTVDGEATEGDAHT
SEQ ID NO: 6: SpyCatcher003-SpyCatcher 003 S59C
VTTLSGLSGEQGPSGDMTTEEDSATHIKFSKRDEDGRELAGATMELRDSSGKTISTWI
SDGHVKDFYLYPGKYTFVETAAPDGYEVATPIEFTVNEDGQVTVDGEATEGDAHTG
SSGSGGGGSGGGGSGVTTLSGLSGEQGPSGDMTTEEDSATHIKFSKRDEDGRELAGA
TMELRDSSGKTISTWICDGHVKDFYLYPGKYTFVETAAPDGYEVATPIEFTVNEDGQ
VTVDGEATEGDAHT
SpyCatcher-Mutations at 127C (SEQ ID NO: 7), F29C (SEQ ID NO: 8), A42C (SEQ ID NO:
9), M44C (SEQ ID NO: 10), W57C (SEQ ID NO: 11), S59C (SEQ ID NO: 1), F75C (SEQ ID
NO: 12), Y84C (SEQ ID NO: 13), A87C (SEQ ID NO: 14), F92C (SEQ ID NO: 15), or V94C
(SEQ ID NO: 16)
VDTLSGLSSEQGQSGDMTIEEDSATHIKFSKRDEDGKELAGATMELRDSSGKTISTWI
SDGQVKDFYLYPGKYTFVETAAPDGYEVATAITFTVNEQGQVTVNGKATKGDAHI
(SEQ ID NO: 47)
SpyCatcher002-Mutations at 127C (SEQ ID NO: 17), F29C (SEQ ID NO: 18), A42C (SEQ
ID NO: 19), M44C (SEQ ID NO: 20), W57C (SEQ ID NO: 21), S59C (SEQ ID NO: 2), F75C
(SEQ ID NO: 22), Y84C (SEQ ID NO: 23), A87C (SEQ ID NO: 24), F92C (SEQ ID NO: 25),
or V94C (SEQ ID NO: 26)
VTTLSGLSGEQGPSGDMTTEEDSATHIKFSKRDEDGRELAGATMELRDSSGKTISTWI
SDGHVKDFYLYPGKYTFVETAAPDGYEVATAITFTVNEQGQVTVNGEATKGDAHT
(SEQ ID NO: 48)
SpyCatcher003-Mutations at 127C (SEQ ID NO: 27), F29C (SEQ ID NO: 28), A42C (SEQ
ID NO: 29), M44C (SEQ ID NO: 30), W57C (SEQ ID NO: 31), S59C (SEQ ID NO: 3), F75C
(SEQ ID NO: 32), Y84C (SEQ ID NO: 33), A87C (SEQ ID NO: 34), F92C (SEQ ID NO: 35),
or V94C (SEQ ID NO: 36)
VTTLSGLSGEQGPSGDMTTEEDSATHIKFSKRDEDGRELAGATMELRDSSGKTISTWI
SDGHVKDFYLYPGKYTFVETAAPDGYEVATPIEFTVNEDGQVTVDGEATEGDAHT
(SEQ ID NO: 49)
SpyCatcher short-Mutations at I6C (SEQ ID NO: 37), F8C (SEQ ID NO: 38), A21C (SEQ
ID NO: 39), M23C (SEQ ID NO: 40), W36C (SEQ ID NO: 41), S38C (SEQ ID NO: 4), F54C
(SEQ ID NO: 42), Y63C (SEQ ID NO: 43), A66C (SEQ ID NO: 44), F71C (SEQ ID NO: 45),
or V73C (SEQ ID NO: 46)
DSATHIKFSKRDEDGKELAGATMELRDSSGKTISTWISDGQVKDFYLYPGKYTFVET
AAPDGYEVATAITFTVNEQGQVTVNG (SEQ ID NO: 50)

Unmodified SpyCatcher, SpyCatcher short, SnoopCatcher, DogCatcher, and SilkCatcher proteins are as follows (isopeptide forming lysines and catalytic glutamic acid (or asparagine in the case of SnoopCatcher) residues are indicated in double underlining:

(SpyCatcher; isopeptide forming lysine at position 31 and catalytic
glutamic acid at position 77)
SEQ ID NO: 47
VDTLSGLSSEQGQSGDMTIEEDSATHIKFSKRDEDGKELAGATMELRDSSGKTISTWI
SDGQVKDFYLYPGKYTFVETAAPDGYEVATAITFTVNEQGQVTVNGKATKGDAHI
(SpyCatcher002)
SEQ ID NO: 48
VTTLSGLSGEQGPSGDMTTEEDSATHIKFSKRDEDGRELAGATMELRD
SSGKTISTWISDGHVKDFYLYPGKYTFVETAAPDGYEVATAITFTVNEQG
QVTVNGEATKGDAHT
(SpyCatcher003)
SEQ ID NO: 49
VTTLSGLSGEQGPSGDMTTEEDSATHIKFSKRDEDGRELAGATMELRDSSGKTISTWI
SDGHVKDFYLYPGKYTFVETAAPDGYEVATPIEFTVNEDGQVTVDGEATEGDAHT
(SpyCatcher short)
SEQ ID NO: 50
DSATHIKFSKRDEDGKELAGATMELRDSSGKTISTWISDGQVKDFYLYPGKYTFVET
AAPDGYEVATAITFTVNEQGQVTVNG
(SnoopCatcher)
SEQ ID NO: 51
KPLRGAVFSLQKQHPDYPDIYGAIDQNGTYQNVRTGEDGKLTFKNLSDGK
YRLFENSEPAGYKPVQNKPIVAFQIVNGEVRDVTSIVPQDIPATYEFTNG
KHYITNEPIPPK
DogCatcher
(SEQ ID NO: 56)
KLGEIEFIKVDKTDKKPLRGAVFSLQKQHPDYPDIYGAIDQNGTYQDVRTGEDGKLT
FTNLSDGKYRLIENSEPPGYKPVQNKPIVSFRIVDGEVRDVTSIVPQ
SilkCatcher
(SEQ ID NO: 58)
SMTYTIELTKHDAATKAVLAGAVYELQDSTGKVIQTGLTTDSQGQLIVKNLRAGDY
QFVETKAPLGYELNTTPVKFTLG

Binding motifs for the SpyCatcher analogs and unmodified SpyCatcher proteins are provided in SEQ ID NOs: 52-54. SEQ ID NO: 55 provides the sequence for the SnoopCatcher binding motif (SnoopTag). SEQ ID NO: 57 (DogTag) provides the binding motif for DogCatcher (SEQ ID NO: 56). SEQ ID NO: 59 (SilkTag) provides the binding motif for SilkCatcher (SEQ ID NO: 58). The amino acids indicated with double underlining cannot be substituted or mutated in analogs of SpyTag, SpyTag002, SpyTag003, SnoopTag, DogTag, or SilkTag analogs that have at least 60% identity to the disclosed sequences.

(SpyTag)
SEQ ID NO: 52
AHIVMVDAYKPTK
(SpyTag002)
SEQ ID NO: 53
VPTIVMVDAYKRYK
(SpyTag003)
SEQ ID NO: 54
RGVPHIVMVDAYKRYK
(SnoopTag)
SEQ ID NO: 55
KLGDIEFIKVNK
DogTag
(SEQ ID NO: 57)
DIPATYEFTDGKHYITNEPIPPK
SilkTag
(SEQ ID NO: 59)
GIKPEVAFQVSQDDVKQPVV

This disclosure will contain reference to “the isopeptide bond forming Lys31 and the catalytic Glu77” within the SpyCatcher, SpyCatcher 002, and SpyCatcher 003 proteins. This designation typically used in the art (see, for example, Zakeri et al., Proc. Natl. Acad. Scie., 2012, 109(12):E690-E697, which is hereby incorporated by reference in its entirety). The isopeptide forming amino acids (lysine) and the catalytic amino acids (glutamic acid or asparagine (in the case of SnoopCatcher)) are identified above by double underlining. In any instance where a mutant or analog of SpyCatcher, SpyCatcher002, SpyCatcher003, SpyCatcher short, SnoopCatcher, DogCatcher, or SilkCatcher is generated, the isopeptide forming amino acid and the catalytic amino acid or each respective protein cannot be substituted or mutated so that a mutant or analog retains the ability to catalyze protein ligation and the phrase “catalyzes protein ligation” indicates that these amino acids have not been substituted or mutated.

Additional embodiments provide SpyCatcher analog comprising any one of SEQ ID NOs: 1-4 or 7-46 or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said mutated SpyCatcher analog having at least 60% sequence identity to SEQ ID NOs: 1-4 or 7-46 catalyzes protein ligation (the isopeptide bond forming Lys31 and the catalytic Glu77 are not mutated/substituted) and the cysteine residue introduced into said mutated SpyCatcher analog is not substituted in said mutated SpyCatcher analog having at least 60% sequence identity to SEQ ID NOs: 1-4 or 7-46.

Yet other embodiments provide a SpyCatcher analog comprising one or more of the mutations in the SpyCatcher protein of SEQ ID NO: 47, said one or more mutations being selected from the group consisting of I27C, F29C, A42C, M44C, W57C, S59C, F75C, Y84C, A87C, F92C, and V94C, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said SpyCatcher analog or said mutated SpyCatcher analog catalyzes protein ligation (the isopeptide bond forming Lys31 and the catalytic Glu77 are not mutated/substituted) and the cysteine residue(s) introduced into said SpyCatcher analog or said mutated SpyCatcher analog is/are not substituted. Where two or more amino acids are exchanged to cysteine residues, these residues can form an intra-chain disulfide bond under oxidizing conditions which then significantly reduces or abolishes the reactivity of SpyCatcher with SpyTag in the absence of Ellmann's reagent or other reagents that would form a disulfide bond with the sulfur atom of the cysteine residue. Upon cleavage of the intra-chain disulfide bond with reducing reagents, SpyCatcher reactivity with SpyTag can be restored.

Additional embodiments provide a SpyCatcher analog comprising one or more of the mutations in the SpyCatcher protein of SEQ ID NO: 47, said one or more mutations being selected from the group consisting of 127, F29, A42, M44, W57, S59, F75, Y84, A87, F92, and V94, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said SpyCatcher analog or said mutated SpyCatcher analog catalyzes protein ligation (the isopeptide bond forming Lys31 and the catalytic Glu77 are not mutated/substituted) and the mutation introduced into said SpyCatcher analog or said mutated SpyCatcher analog is/are not substituted. For these SpyCatcher analogs, the mutations that are introduced are: a) unnatural amino acids such as photocaged cysteines (e.g., S—[(R,S)-1-{4′,5′-Dimethoxy-2′-nitrophenyl}ethyl]-L-cysteine or (2R)-2-Amino-3-{[({[1-(6-nitrobenzo[d][1,3]dioxol-5-yl)ethoxy]carbony}amino)methyl]thio}propanoic acid, photocaged lysines (e.g., (S)-2-Amino-6-((((7-hydroxy-2-oxo-2H-chromen-4-yl)methoxy)carbonyl)amino)hexanoic acid), photocaged tyrosine (e.g., (2S)-2-Amino-3-(4-(1-(6-nitrobenzo[d][1,3]dioxol-5-yl)ethoxy)phenyl)propanoic acid), chemically caged lysines (e.g., N6-(((4-azidobenzyl)oxy)carbonyl)-L-lysine), or combinations of these unnatural amino acids; or b) cysteine residues in combination with unnatural amino acids, such as photocaged cysteines (e.g., S—[(R,S)-1-{4′,5′-Dimethoxy-2′-nitrophenyl}ethyl]-L-cysteine or (2R)-2-Amino-3-{[({[1-(6-nitrobenzo[d][1,3]dioxol-5-yl)ethoxy]carbonyl}amino)methyl]thio}propanoic acid, photocaged lysines (e.g., (S)-2-Amino-6-((((7-hydroxy-2-oxo-2H-chromen-4-yl)methoxy)carbonyl)amino)hexanoic acid), photocaged tyrosine (e.g., (2S)-2-Amino-3-(4-(1-(6-nitrobenzo[d][1,3]dioxol-5-yl)ethoxy)phenyl)propanoic acid), chemically caged lysines (e.g., N6-(((4-azidobenzyl)oxy)carbonyl)-L-lysine), or combinations of these unnatural amino acids. Other examples include those discussed in Yamaguchi App. Sci., 2022, 12:3750 (doi.org/10.3390/app12083750), such as caged aspartic acid, caged serine, caged glycine, caged tyrosine, caged cysteine, and caged lysine caged phosphoserine and caged phosphotyrosine. Additionally, amino acids with selectively removable bulky substituents can be introduced to SpyCatcher and other proteins of interest by peptide synthesis. For example, a cysteine caged with 6-bromo-7-hydroxy-3-methylcoumarin-4-ylmethyl (Mahmoodi et al., 2016, Org. Biomol. Chem., 14:8289) can be introduced into the SpyCatcher analog at one or more of the positions disclosed within this paragraph.

Yet other embodiments provide a SpyCatcher analog comprising one or more of the mutations in the SpyCatcher protein of SEQ ID NO: 48, said one or more mutations being selected from the group consisting of 127C, F29C, A42C, M44C, W57C, S59C, F75C, Y84C, A87C, F92C, and V94C, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said SpyCatcher analog or said mutated SpyCatcher analog catalyzes protein ligation (the isopeptide bond forming Lys31 and the catalytic Glu77 are not mutated/substituted) and the cysteine residue(s) introduced into said SpyCatcher analog or said mutated SpyCatcher analog is/are not substituted in said SpyCatcher analog or said polypeptide. Where two or more amino acids are exchanged to cysteine residues, these residues can form an intra-chain disulfide bond under oxidizing conditions which then significantly reduces or abolishes the reactivity of SpyCatcher with SpyTag in the absence of Ellmann's reagent or other reagents that would form a disulfide bond with the sulfur atom of the cysteine residue. Upon cleavage of the intra-chain disulfide bond with reducing reagents, SpyCatcher reactivity with SpyTag can be restored.

Additional embodiments provide a SpyCatcher analog comprising one or more of the mutations in the SpyCatcher protein of SEQ ID NO: 48, said one or more mutations being selected from the group consisting of 127, F29, A42, M44, W57, 559, F75, Y84, A87, F92, and V94, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said SpyCatcher analog or said mutated SpyCatcher analog catalyzes protein ligation (the isopeptide bond forming Lys31 and the catalytic Glu77 are not mutated/substituted) and the mutation introduced into said SpyCatcher analog or said mutated SpyCatcher analog is/are not substituted. For these SpyCatcher analogs, the mutations that are introduced are: a) unnatural amino acids such as photocaged cysteines (e.g., S—[(R,S)-1-{4′,5′-Dimethoxy-2′-nitrophenyl}ethyl]-L-cysteine or (2R)-2-Amino-3-{[({[1-(6-nitrobenzo[d]1[1,3]dioxol-5-yl)ethoxy]carbonyl}amino)methyl]thio}propanoic acid, photocaged lysines (e.g., (S)-2-Amino-6-((((7-hydroxy-2-oxo-2H-chromen-4-yl)methoxy)carbonyl)amino)hexanoic acid), photocaged tyrosine (e.g., (2S)-2-Amino-3-(4-(1-(6-nitrobenzo[d][1,3]dioxol-5-yl)ethoxy)phenyl)propanoic acid), chemically caged lysines (e.g., N6-(((4-azidobenzyl)oxy)carbonyl)-L-lysine), or combinations of these unnatural amino acids; or b) cysteine residues in combination with unnatural amino acids, such as photocaged cysteines (e.g., S—[(R,S)-1-{4′,5′-Dimethoxy-2′-nitrophenyl}ethyl]-L-cysteine or (2R)-2-Amino-3-{[({[1-(6-nitrobenzo[d][1,3]dioxol-5-yl)ethoxy]carbonyl}amino)methyl]thio}propanoic acid, photocaged lysines (e.g., (S)-2-Amino-6-((((7-hydroxy-2-oxo-2H-chromen-4-yl)methoxy)carbonyl)amino)hexanoic acid), photocaged tyrosine (e.g., (2S)-2-Amino-3-(4-(1-(6-nitrobenzo[d][1,3]dioxol-5-yl)ethoxy)phenyl)propanoic acid), chemically caged lysines (e.g., N6-(((4-azidobenzyl)oxy)carbonyl)-L-lysine), or combinations of these unnatural amino acids. Additionally, amino acids with selectively removable bulky substituents can be introduced to SpyCatcher and other proteins of interest by peptide synthesis. For example, a cysteine caged with 6-bromo-7-hydroxy-3-methylcoumarin-4-ylmethyl (Mahmoodi et al., 2016, Org. Biomol. Chem., 14:8289) can be introduced into the SpyCatcher analog at one or more of the positions disclosed within this paragraph.

Yet other embodiments provide a SpyCatcher analog comprising one or more of the mutations in the SpyCatcher protein of SEQ ID NO: 49, said one or more mutations being selected from the group consisting of I27C, F29C, A42C, M44C, W57C, S59C, F75C, Y84C, A87C, F92C, and V94C, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said SpyCatcher analog or said mutated SpyCatcher analog catalyzes protein ligation (the isopeptide bond forming Lys31 and the catalytic Glu77 are not mutated/substituted) and the cysteine residue(s) introduced into said SpyCatcher analog or said mutated SpyCatcher analog is/are not substituted in said SpyCatcher analog or said polypeptide. Where two or more amino acids are exchanged to cysteine residues, these residues can form an intra-chain disulfide bond under oxidizing conditions which then significantly reduces or abolishes the reactivity of SpyCatcher with SpyTag in the absence of Ellmann's reagent or other reagents that would form a disulfide bond with the sulfur atom of the cysteine residue. Upon cleavage of the intra-chain disulfide bond with reducing reagents, SpyCatcher reactivity with SpyTag can be restored.

Additional embodiments provide a SpyCatcher analog comprising one or more of the mutations in the SpyCatcher protein of SEQ ID NO: 49, said one or more mutations being selected from the group consisting of 127, F29, A42, M44, W57, S59, F75, Y84, A87, F92, and V94, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said SpyCatcher analog or said mutated SpyCatcher analog catalyzes protein ligation (the isopeptide bond forming Lys31 and the catalytic Glu77 are not mutated/substituted) and the mutation introduced into said SpyCatcher analog or said mutated SpyCatcher analog is/are not substituted. For these SpyCatcher analogs, the mutations that are introduced are: a) unnatural amino acids such as photocaged cysteines (e.g., S—[(R,S)-1-{4′,5′-Dimethoxy-2′-nitrophenyl}ethyl]-L-cysteine or (2R)-2-Amino-3-{[({[1-(6-nitrobenzo[d][1,3]dioxol-5-yl)ethoxy]carbonyl}amino)methyl]thio}propanoic acid, photocaged lysines (e.g., (S)-2-Amino-6-((((7-hydroxy-2-oxo-2H-chromen-4-yl)methoxy)carbonyl)amino)hexanoic acid), photocaged tyrosine (e.g., (2S)-2-Amino-3-(4-(1-(6-nitrobenzo[d][1,3]dioxol-5-yl)ethoxy)phenyl)propanoic acid), chemically caged lysines (e.g., N6-(((4-azidobenzyl)oxy)carbonyl)-L-lysine), or combinations of these unnatural amino acids; or b) cysteine residues in combination with unnatural amino acids, such as photocaged cysteines (e.g., S—[(R,S)-1-{4′,5′-Dimethoxy-2′-nitrophenyl}ethyl]-L-cysteine or (2R)-2-Amino-3-{[({[1-(6-nitrobenzo[d][1,3]dioxol-5-yl)ethoxy]carbonyl}amino)methyl]thio}propanoic acid, photocaged lysines (e.g., (S)-2-Amino-6-((((7-hydroxy-2-oxo-2H-chromen-4-yl)methoxy)carbonyl)amino)hexanoic acid), photocaged tyrosine (e.g., (2S)-2-Amino-3-(4-(1-(6-nitrobenzo[d][1,3]dioxol-5-yl)ethoxy)phenyl)propanoic acid), chemically caged lysines (e.g., N6-(((4-azidobenzyl)oxy)carbonyl)-L-lysine), or combinations of these unnatural amino acids. Additionally, amino acids with selectively removable bulky substituents can be introduced to SpyCatcher and other proteins of interest by peptide synthesis. For example, a cysteine caged with 6-bromo-7-hydroxy-3-methylcoumarin-4-ylmethyl (Mahmoodi et al., 2016, Org. Biomol. Chem., 14:8289) can be introduced into the SpyCatcher analog at one or more of the positions disclosed within this paragraph.

Yet other embodiments provide a SpyCatcher analog comprising one or more of the mutations in the SpyCatcher protein of SEQ ID NO: 50, said one or more mutations being selected from the group consisting of I6C, F8C, A21C, M23C, W36C, S38C, F54C, Y63C, A66C, F71C, and V73C, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said SpyCatcher analog or said mutated SpyCatcher analog catalyzes protein ligation (the isopeptide bond forming Lys31 and the catalytic Glu77 are not mutated/substituted) and the cysteine residue(s) introduced into said SpyCatcher analog or said mutated SpyCatcher analog is/are not substituted in said SpyCatcher analog or said polypeptide. Where two or more amino acids are exchanged to cysteine residues, these residues can form an intra-chain disulfide bond under oxidizing conditions which then significantly reduces or abolishes the reactivity of SpyCatcher with SpyTag in the absence of Ellmann's reagent or other reagents that would form a disulfide bond with the sulfur atom of the cysteine residue. Upon cleavage of the intra-chain disulfide bond with reducing reagents, SpyCatcher reactivity with SpyTag can be restored.

Additional embodiments provide a SpyCatcher analog comprising one or more of the mutations in the SpyCatcher protein of SEQ ID NO: 50, said one or more mutations being selected from the group consisting of 16, F8, A21, M23, W36, S38, F54, Y63, A66, F71, and V73, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said SpyCatcher analog or said mutated SpyCatcher analog catalyzes protein ligation (the isopeptide bond forming Lys31 and the catalytic Glu77 are not mutated/substituted) and the mutation introduced into said SpyCatcher analog or said mutated SpyCatcher analog is/are not substituted. For these SpyCatcher analogs, the mutations that are introduced are: a) unnatural amino acids such as photocaged cysteines (e.g., S—[(R,S)-1-{4′,5′-Dimethoxy-2′-nitrophenyl}ethyl]-L-cysteine or (2R)-2-Amino-3-{[({[1-(6-nitrobenzo[d][1,3]dioxol-5-yl)ethoxy]carbonyl}amino)methyl]thio}propanoic acid, photocaged lysines (e.g., (S)-2-Amino-6-((((7-hydroxy-2-oxo-2H-chromen-4-yl)methoxy)carbonyl)amino)hexanoic acid), photocaged tyrosine (e.g., (2S)-2-Amino-3-(4-(1-(6-nitrobenzo[d][1,3]dioxol-5-yl)ethoxy)phenyl)propanoic acid), chemically caged lysines (e.g., N6-(((4-azidobenzyl)oxy)carbonyl)-L-lysine), or combinations of these unnatural amino acids; or b) cysteine residues in combination with unnatural amino acids, such as photocaged cysteines (e.g., S—[(R,S)-1-{4′,5′-Dimethoxy-2′-nitrophenyl}ethyl]-L-cysteine or (2R)-2-Amino-3-{[({[1-(6-nitrobenzo[d][1,3]dioxol-5-yl)ethoxy]carbonyl}amino)methyl]thio}propanoic acid, photocaged lysines (e.g., (S)-2-Amino-6-((((7-hydroxy-2-oxo-2H-chromen-4-yl)methoxy)carbonyl)amino)hexanoic acid), photocaged tyrosine (e.g., (2S)-2-Amino-3-(4-(1-(6-nitrobenzo[d][1,3]dioxol-5-yl)ethoxy)phenyl)propanoic acid), chemically caged lysines (e.g., N6-(((4-azidobenzyl)oxy)carbonyl)-L-lysine), or combinations of these unnatural amino acids. Additionally, amino acids with selectively removable bulky substituents can be introduced to SpyCatcher and other proteins of interest by peptide synthesis. For example, a cysteine caged with 6-bromo-7-hydroxy-3-methylcoumarin-4-ylmethyl (Mahmoodi et al., 2016, Org. Biomol. Chem., 14:8289) can be introduced into the SpyCatcher analog at one or more of the positions disclosed within this paragraph.

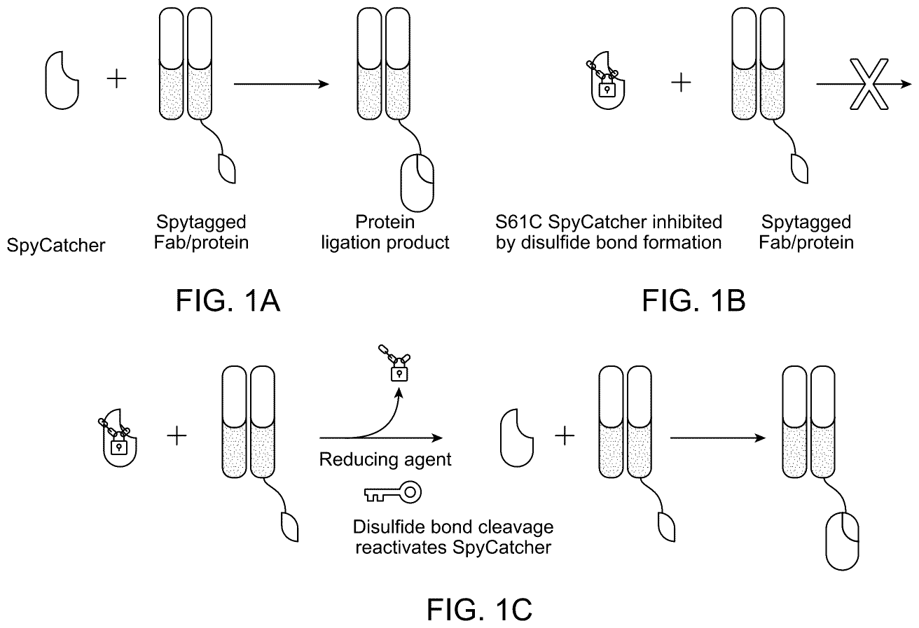

The SpyCatcher analogs are capable of being “locked” by molecules that modify the cysteine residue that has been introduced into the SpyCatcher protein (forming “SpyLock” proteins). SpyCatcher analogs comprising the cysteine residue that has been modified by a molecule are referred to as “SpyLock” proteins. For example, disulfide-forming molecules such as Ellmann's reagent (5,5′-dithio-bis-(2-nitrobenzoic acid), DTNB) or HPDP-biotin (N-[6-(biotinamido)hexyl]-3′-(2′-pyridyldithio)propionamide) significantly reduce or abolish the activity of the SpyCatcher analogs with respect to the isopeptide bond formation with SpyTag or its analogs SpyTag2 or SpyTag3. Alternatively, 3-nitro-2-pyridylsulfenyl (Npys) activated peptides, e.g., Npys-Cys-StrepTag2 can be used to react with the thiol group introduced in to the SpyCatcher analog. Alternatively, photocleavable isoxazolinium reagents can be used to “lock” a SpyCatcher analog (forming a SpyLock protein). SpyCatcher-SpyTag reactivity can be restored by treatment with chemical compounds which remove the locking agent, for example with reducing agents that cleave the disulfide bond, or by removing the photocleavable blocking group using light as discussed above. Thus, the modified SpyCatcher proteins can be “locked” or “unlocked” depending on the application (see FIG. 1). Accordingly, another aspect of the invention relates to “locked” SpyCatcher analogs in which the cysteine residue has been modified with Ellmann's reagent, HPDP-biotin or another molecule that reversibly forms a bond with cysteine in the SpyCatcher analog.

A “locked” SpyCatcher analog (which can also be referred to as a “SpyLock protein”) is an analog in which the cysteine that has been introduced into the SpyCatcher analog has formed a chemical bond with a blocking molecule, such as HPDP-biotin, Ellmann's reagent or a photocleavable reagent as disclosed above. An “unlocked” SpyCatcher analog is an analog in which the thiol (sulfhydryl) group is free.

Another option is the translational incorporation of unnatural amino acids, such as S—[(R,S)-1-{4′,5′-Dimethoxy-2′-nitrophenyl}ethyl]-L-cysteine, or to use modified SpyCatcher (e.g., by 6-bromo-7-hydroxy-3-methylcoumarin-4-ylmethyl cysteine) generated by peptide synthesis to form a SpyCatcher analog. Such a SpyCatcher analog is “locked” and can be unlocked, e.g., by irradiation and photocleavage. A “locked” SpyCatcher analog generated with unnatural amino acids is an analog which does not react with the SpyTag whereas the “unlocked” SpyCatcher has reinstalled the activity to SpyTag.

The subject disclosure also provides another SpyCatcher analog in which the reactive lysine is “locked” with, for example, a photocaged lysine (e.g., (S)-2-Amino-6-((((7-hydroxy-2-oxo-2H-chromen-4-yl)methoxy)carbonyl)amino)hexanoic acid) or a chemically caged lysine (e.g., N6-(((4-azidobenzyl)oxy)carbonyl)-L-lysine). A photocaged lysine that is introduced into SpyCatcher, SpyCatcher 002, or SpyCatcher003 would result in a SpyLock protein that is unable to catalyze the formation of a peptide bond. After photocleavage of the blocking group, catalytic activity of the SpyCatcher analog would be restored.

### Fusion Proteins

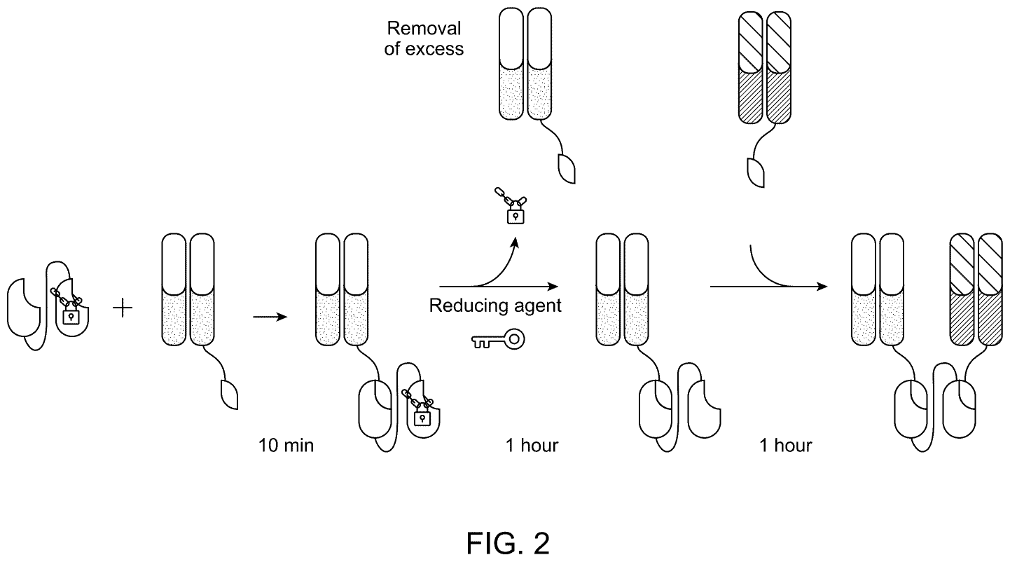

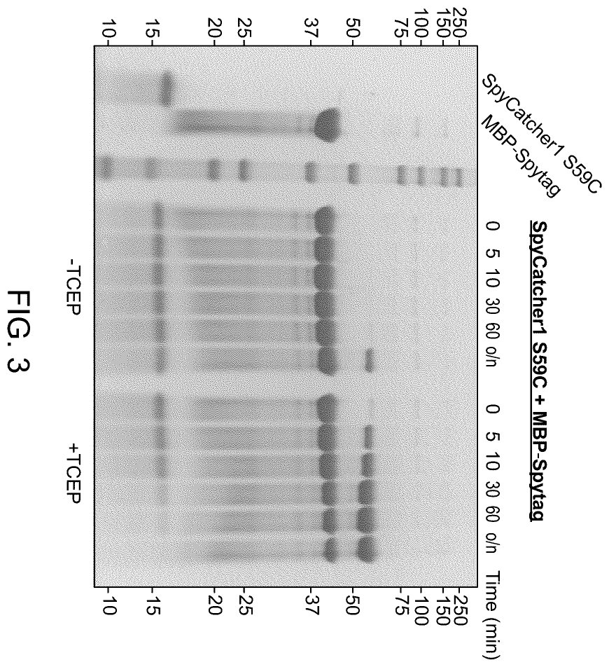

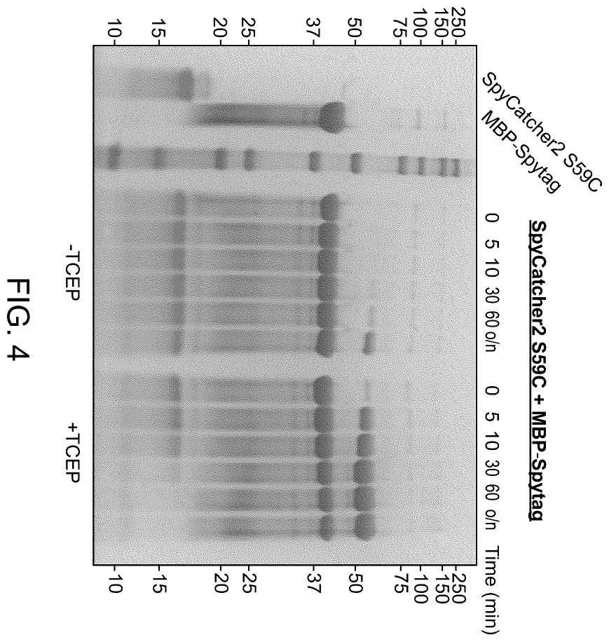

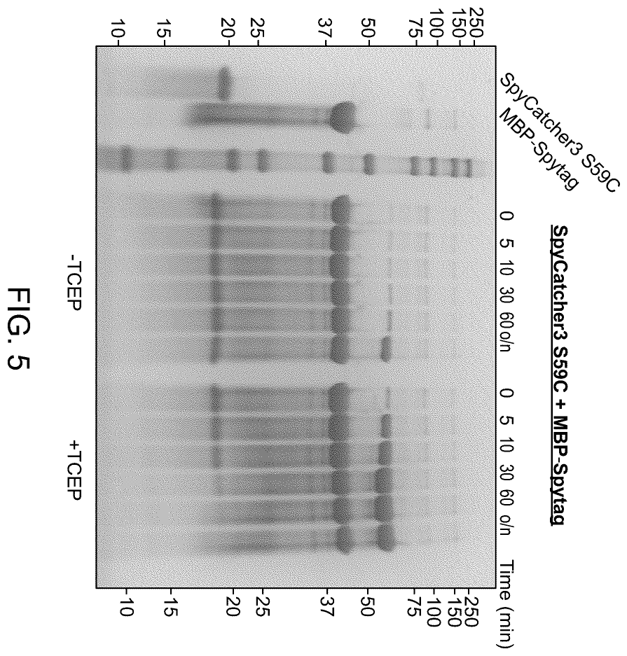

In another aspect of the invention, fusion proteins comprising at least one SpyCatcher analog are provided. In the context of the disclosed fusion proteins, the SpyCatcher analogs, unmodified SpyCatcher proteins, and SnoopCatcher may be referred to as “elements” of the fusion protein, “elements” of a BiCatcher, “elements” of a HeteroCatcher, or “elements” of a MultiCatcher. For example, a fusion protein comprising at least one SpyCatcher analog and at least one unmodified SpyCatcher (e.g., a “modified BiCatcher” such as SpyCatcher analog-SpyCatcher) or a “modified MultiCatcher” that includes at least one modified SpyCatcher that is “unlocked” or “locked” and contains 2 or more additional elements (see FIG. 2) is provided. The elements of the fusion protein may be joined by a linker sequence. In such an embodiment, the modified BiCatcher or modified MultiCatcher can be “locked” with Ellmann's reagent (DTNB), HPDP-biotin, Npys-activated affinity tag peptides, e.g., Npys-Cys-StrepTag2, a photocleavable chemical group as disclosed above (e.g., 6-bromo-7-hydroxy-3-methylcoumarin-4-ylmethyl (mBhct) or a photocleavable isoxazolinium reagent) or by incorporation of a chemically caged unnatural amino acid into the SpyCatcher analog. As with an unlocked SpyCatcher analog, an “unlocked BiCatcher” or “unlocked MultiCatcher” is a fusion protein in which the thiol group of the cysteine introduced into the SpyCatcher analog is free and available to form a disulfide bond with another group, such as a thiol group in Ellmann's reagent or HPDP-biotin or in which the caging group of the unnatural amino acid has be cleaved, e.g., by photocleavage.

The fusion protein can also be composed of different elements, such as a SpyCatcher analog and one or more element selected from SnoopCatcher, DogCatcher, and SilkCatcher that is orthogonal to SpyCatcher (e.g., a “HeteroCatcher”). In such embodiments, the HeteroCatcher comprises at least one SpyCatcher analog and another protein capable of protein ligation, such as SnoopCatcher. A HeteroCatcher can, in addition to one or more orthogonal element, further comprise additional unmodified SpyCatcher proteins. The elements of the “HeteroBiCatcher” can be attached to each other in random or sequential order, e.g., SpyCatcher analog-SpyCatcher-SnoopCatcher-SnoopCatcher, SnoopCatcher-SnoopCatcher-SpyCatcher-SpyCatcher analog, SpyCatcher analog-SnoopCatcher-SpyCatcher-SnoopCatcher, SpyCatcher-SnoopCatcher-SnoopCatcher-SpyCatcher analog, or SnoopCatcher-SpyCatcher analog-SpyCatcher-SnoopCatcher, etc.

As discussed above, the SpyCatcher analog element of the fusion protein is a SpyCatcher analog comprising any one of SEQ ID NOs: 1-4 or 7-46 or a polypeptide having at least 60%, 65%, 70%, 75%, 80%, 85%, 90%, 91%, 92%, 93%, 94%, 95%, 96%, 97%, 98%, or 99% identity sequence identity to a SpyCatcher analog comprising any one of SEQ ID NOs: 1-4 or 7-46, with the proviso that said SpyCatcher analog having at least 60% sequence identity to SEQ ID NOs: 1-4 or 7-46 catalyzes protein ligation (the isopeptide bond forming Lys31 and the catalytic Glu77 are not mutated/substituted) and the at least one cysteine residue and/or unnatural amino acid introduced into said SpyCatcher analog is not substituted in said polypeptide having at least 60% sequence identity to SEQ ID NOs: 1-4 or 7-46. The other element or elements of the fusion protein can be non-mutated SpyCatcher or SnoopCatcher, DogCatcher, or SilkCatcher proteins that have at least 60%, 65%, 70%, 75%, 80%, 85%, 90%, 91%, 92%, 93%, 94%, 95%, 96%, 97%, 98%, or 99% sequence identity or a SpyCatcher or SnoopCatcher protein as disclosed in any one of SEQ ID NOs: 47-51 with the proviso that said non-mutated SpyCatcher or SnoopCatcher protein having at least 60% sequence identity to any one of SEQ ID NOs: 47-51 catalyzes protein ligation (the isopeptide bond forming Lys31 and the catalytic Glu77 are not mutated/substituted in the case of SpyCatcher).

### Polypeptides and Antigen Binding Proteins

In various embodiments, a polypeptide (e.g., a fluorescent protein, an enzyme, an effector protein, or an antigen binding fragment) comprising a first binding motif (such as a SpyTag or SnoopTag), and fusion protein comprising at least one modified SpyCatcher analog as disclosed herein are provided. In some embodiments, an antigen binding protein that comprises two or more antigen binding fragments, each comprising a first binding motif, and fusion protein comprising at least one modified SpyCatcher analog are also provided. The first binding motif joined to the first polypeptide (e.g., an antigen binding fragment) may, optionally, be joined by one or more linker sequence and may be SnoopTag, SpyTag, SpyTag002, or SpyTag003. The first binding motif of a polypeptide (e.g., a first antigen binding fragment) are covalently conjugated to the unmodified SpyCatcher or SnoopCatcher of a locked BiCatcher, a locked HeteroCatcher, or a locked MultiCatcher. After covalent conjugation of the first binding motif to the unmodified SpyCatcher or SnoopCatcher of the locked BiCatcher, locked HeteroCatcher, or the locked MultiCatcher, excess polypeptide (e.g., first antigen binding fragment) is removed. Alternatively, a 1:1 ratio (unmodified SpyCatcher:SpyTagged first antigen binding fragment) can be used for the first coupling reaction to avoid any purification steps. The polypeptide or antigen binding fragment covalently conjugated to the locked BiCatcher, locked HeteroCatcher or locked MultiCatcher is either chemically uncaged, e.g., by treatment with a reducing agent, such as dithiothreitol (DTT) or photo-uncaged to unlock the BiCatcher, HeteroCatcher or MultiCatcher. A second polypeptide (e.g., an antigen binding fragment) comprising a first binding motif, such as SpyTag, SpyTag002, or SpyTag003, is then covalently conjugated to the unlocked SpyCatcher analog present in the unlocked BiCatcher, unlocked HeteroCatcher, or the unlocked MultiCatcher via protein ligation.

In various embodiments, the antigen binding protein can further comprise a detectable label. Exemplary detectable labels include, but are not limited to, a fluorophore, a fluorescent protein such as green fluorescent protein (GFP), biotin, an enzyme such as horseradish peroxidase (HRP) or other peroxidases, alkaline phosphatase, luciferase, and a split fluorescent protein (e.g., split GFP) or enzymes (e.g., NanoLuc® Binary Technology from Promega). Exemplary fluorophores include, but are not limited to, Alexa dyes (e.g., Alexa 350, Alexa 430, Alexa 488, etc.), AMCA, BODIPY 630/650, BODIPY 650/665, BODIPY-FL, BODIPY-R6G, BODIPY-TMR, BODIPY-TRX, Cascade Blue, Cy2, Cy3, Cy5, Cy5.5, Cy7, Cy7.5, Dylight dyes (Dylight405, Dylight488, Dylight549, Dylight550, Dylight 649, Dylight680, Dylight750, Dylight800), 6-FAM, fluorescein, FITC, HEX, 6-JOE, Oregon Green 488, Oregon Green 500, Oregon Green 514, Pacific Blue, REG, Rhodamine Green, Rhodamine Red, ROX, R-Phycoerythrin (R-PE), Starbright Dyes (e.g., Starbright Blue 580, Starbright Blue 700, Starbright violet 440 etc.), TAMRA, TET, Tetramethylrhodamine, Texas Red, and TRITC.

A “linker sequence” or “linker” herein refers to a peptide or polypeptide containing two or more amino acid residues joined by peptide bond(s) that provides increased rotational freedom for two polypeptides linked thereby than the two linked polypeptides would have in the absence of the linker. Such rotational freedom allows each component of the fusion protein to interact with its intended target without hindrance. Generally, these linkers are mixtures of glycine and serine, such as -(GGGS)n-, where n is 1, 2, 3, 4, or 5. Other suitable peptide/polypeptide linker sequence optionally include naturally occurring or non-naturally occurring peptides or polypeptides. Peptide linker sequences are at least 2 amino acids in length. Optionally the peptide or polypeptide domains are flexible peptides or polypeptides. Exemplary flexible peptides/polypeptides include, but are not limited to, the amino acid sequences (Gly)n, where n is an integer between 2 and 50, Gly-Ser, Gly-Ser-Gly-Ser, Ala-Ser, Gly-Gly-Gly-Ser, Gly₄-Ser, (Gly₄-Ser)₂, (Gly₄-Ser)₃, (Gly₄-Ser)₄, (Gly₄-Ser)₂-Gly-Ala-Gly-Ser-Gly₄-Ser, Gly-(Gly₄-Ser)₂, Gly₄-Ser-Gly, Gly-Ser-Gly₂ and Gly-Ser-Gly₂-Ser. Other suitable peptide linker domains optionally include the TEV linker ENLYFQG (SEQ ID NO: 61), a linear epitope recognized by the Tobacco Etch virus protease. Exemplary peptides/polypeptides include, but are not limited to, GSENLYFQGSG (SEQ ID NO: 62). Similarly, other protease cleavage sequences can be used, e.g., PreScission protease. Other suitable peptide linker sequences include helix forming linkers such as Ala-(Glu-Ala-Ala-Ala-Lys)n-Ala (n=1-5). In some embodiments, the linker sequence is a GAP (Gly Ala Pro) sequence. In some embodiments, a sequence of 1 to 50 amino acid residues can be used as a linker. In some embodiments such linkers are soluble, flexible, and protease resistant (i.e., expression of a polypeptide having the linker in a host cell occurs without cleavage of the linker by a protease). As indicated above, the phrase “joined by a linker sequence” permits the use of one or more linker sequences to connect two or more binding motifs, a binding motif and a polypeptide or a binding motif and an antigen binding fragment. Where a plurality of linker sequences are used to join binding motifs, to join antigen binding fragments to a binding motif, or to join a polypeptide and a binding motif, the linker sequences can be the same or different.

Methods of producing antigen binding proteins are also provided. In an embodiment, the method comprises contacting a first antigen binding fragment comprising a first binding motif such as SpyTag, SpyTag002, or SpyTag003, with a locked BiCatcher or locked MultiCatcher. The conditions for contacting the first antigen binding fragment and the locked BiCatcher or locked MultiCatcher are such that a covalent bond is formed between the first binding motif and the unmodified SpyCatcher of the locked BiCatcher or locked MultiCatcher via protein ligation. After removal of excess first antigen binding fragment or using a 1:1 ratio without purification step, a second antigen binding fragment comprising a binding motif such as SpyTag, SpyTag002, or SpyTag003 is then added to the antigen binding fragment ligated to the locked BiCatcher or locked MultiCatcher. The locked BiCatcher or locked MultiCatcher is then treated with a reducing agent, such as DTT, to unlock the BiCatcher or MultiCatcher. Alternatively, the BiCatcher or MultiCatcher is first unlocked and then the second antigen binding fragment comprising a binding motif such as SpyTag is added.

The term “protein ligation” as used herein refers to site-specific covalent bond formation, either spontaneously or with the help of an enzyme, between a binding motif such as modified SpyCatcher as disclosed herein (SEQ ID NOs: 1-46) SpyCatcher, SpyCatcher short, SpyCatcher002, SpyCatcher003, or SnoopCatcher. Also, as discussed throughout this disclosure, protein ligation occurs between specific combinations of binding motifs, for example, between SpyTag (SEQ ID NO: 52), SpyTag002 (SEQ ID NO: 53), or SpyTag003 (SEQ ID NO: 54) and SpyCatcher (SEQ ID NO: 47), SpyCatcher short (SEQ ID NO: 50), SpyCatcher002 (SEQ ID NO: 48), or SpyCatcher003 (SEQ ID NO: 49) or the SpyCatcher analogs of any one of SEQ ID NOs: 1-46.

Peptides having at least 60%, 65%, 70%, 75%, 80%, 85%, 90%, 91%, 92%, 93%, 94%, 95%, 96%, 97%, 98%, or 99% sequence identity to any one of SEQ ID NOs: 52-55, 57, and 59 are referred to as SpyTag, SpyTag002, SpyTag003, SnoopTag, DogTag, or SilkTag analogs and form an isopeptide bond with a corresponding SpyCatcher, SpyCatcher002, SpyCatcher003, SnoopCatcher, DogCatcher, or SilkCatcher protein or a SpyCatcher, SpyCatcher002, SpyCatcher003, SnoopCatcher, DogCatcher, or SilkCatcher analog (the Asp residue is not mutated in the SpyTag analog). Analogs of SEQ ID NOs: 55, 57, and 59 may be referred to as SnoopTag, DogTag, and SilkTag analogs respectively. Those amino acids that cannot be substituted or mutated in SpyTag, SpyTag002, SpyTag003, SnoopTag, DogTag, or SilkTag analogs are indicated by double underlining in SEQ ID NOs: 52-55, 57 and 59.

Therefore, to produce an antigen binding protein, a first binding motif present in a first antigen binding fragment (e.g., at the C-terminus, the N-terminus, or embedded within the amino acid sequence) is capable of forming a covalent bond via protein ligation to a SpyCatcher or an unlocked SpyCatcher in a first fusion protein. For example, if a first binding motif present in a first antigen binding fragment is a SpyTag, SpyTag002 or SpyTag003, the corresponding second binding motif is a SpyCatcher protein or a SpyCatcher analog present in the BiCatcher, HeteroCatcher, or MultiCatcher fusion protein.

The expression of the above proteins can be carried out in suitable host cells, including prokaryotic cells, such as Escherichia coli or eukaryotic cells, such as yeast or mammalian cells, e.g., CHO cells. In certain embodiments, the disclosed proteins can be produced in protease deficient prokaryotic cells, such as protease deficient E. coli. In various embodiments, the protease deficient prokaryotic cells (e.g., protease deficient E. coli cells) are periplasmic protease deficient.

Appropriate conditions, such as buffer components, pH, and temperature can be provided for optimal conjugation via SpyTag/SpyCatcher and SnoopTag/SnoopCatcher systems. The antigen binding protein so produced can be used as is or further purified before use. Such purification can be performed by size exclusion chromatography, affinity chromatography or other chromatographic or other separation techniques known in the art.

### Nucleic Acid Constructs

Also provided are nucleic acid constructs that encode for SpyCatcher analogs and nucleic acid constructs that encode fusion proteins comprising at least one SpyCatcher analog as disclosed herein. Such nucleic acids can be present in an expression vector in an appropriate host cell. As discussed below, the host cells can be prokaryotic or eukaryotic.

Accordingly, in an embodiment, a nucleic acid construct comprises a nucleic acid construct comprising a polynucleotide encoding a SpyCatcher analog, a modified BiCatcher comprising at least one SpyCatcher analog, or a modified MultiCatcher comprising at least one SpyCatcher analog. The elements of the modified BiCatcher or modified MultiCatcher may, optionally, be joined by a linker sequence.

The nucleic acid constructs are typically introduced into various vectors. The vectors of the present invention generally comprise transcriptional or translational control sequences required for expressing the fusion proteins. Suitable transcription or translational control sequences include but are not limited to replication origin, promoter, enhancer, repressor binding regions, transcription initiation sites, ribosome binding sites, translation initiation sites, and termination sites for transcription and translation.

The origin of replication (generally referred to as an ori sequence) permits replication of the vector in a suitable host cell. The choice of on will depend on the type of host cells and/or genetic packages that are employed. Where the host cells are prokaryotes, the expression 20 vector typically comprises ori sequences directing autonomous replication of the vector within the prokaryotic cells. Preferred prokaryotic on is capable of directing vector replication in bacterial cells. Non-limiting examples of this class of ori include pMB1, pUC, as well as other E. coli origins.

In the eukaryotic system, higher eukaryotes contain multiple origins of DNA replication, but the on sequences are not clearly defined. The suitable origins of replication for mammalian vectors are normally from eukaryotic viruses. Preferred eukaryotic ori include, but are not limited to, SV40 ori, EBV ori, or HSV ori.

As used herein, a “promoter” is a DNA region capable under certain conditions of binding RNA polymerase and initiating transcription of a coding region located downstream (in the 3′ direction) from the promoter. It can be constitutive or inducible. In general, the promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding sites responsible for the binding of RNA polymerase. Eukaryotic promoters will often, but not always, contain “TATA” boxes and “CAT” boxes.

The choice of promoters will largely depend on the host cells in which the vector is introduced. For prokaryotic cells, a variety of robust promoters are known in the art. Preferred promoters are lac promoter, Trc promoter, T7 promoter and PBAD promoter. Normally, to obtain expression of exogenous sequence in multiple species, the prokaryotic promoter can be placed immediately after the eukaryotic promoter, or inside an intron sequence downstream of the eukaryotic promoter.

Suitable promoter sequences for eukaryotic cells include the promoters for 3-phosphoglycerate kinase, or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other promoters, which have the additional advantage of transcription controlled by growth conditions, are the promoter regions for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Preferred promoters for mammalian cells are SV40 promoter, CMV promoter, 3-actin promoter and their hybrids. Preferred promoters for yeast cell includes but is not limited to GAL 10, GAL I, TEFI in S. cerevisiae, and GAP, AOX1 in P. pastoris.

In constructing the subject vectors, the termination sequences associated with the protein coding sequence can also be inserted into the 3′ end of the sequence desired to be transcribed to provide polyadenylation of the mRNA and/or transcriptional termination signal. The terminator sequence preferably contains one or more transcriptional termination sequences (such as polyadenylation sequences) and may also be lengthened by the inclusion of additional DNA sequence so as to further disrupt transcriptional read-through.

In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins, e.g., ampicillin, kanamycin, neomycin, zeocin, G418, methotrexate, etc.; or (b) complement auxotrophic deficiencies. The choice of the proper marker gene will depend on the host cell, and appropriate genes for different hosts are known in the art.

In one embodiment, the expression vector is a shuttle vector, capable of replicating in at least two unrelated host systems. In order to facilitate such replication, the vector generally contains at least two origins of replication, one effective in each host system. Typically, shuttle vectors are capable of replicating in a eukaryotic host system and a prokaryotic host system. This enables detection of protein expression in the eukaryotic host (the expression cell type) and amplification of the vector in the prokaryotic host (the amplification cell type). Preferably, one origin of replication is derived from SV40 or 2p and one is derived from pUC, although any suitable origin known in the art may be used provided it directs replication of the vector. Where the vector is a shuttle vector, the vector preferably contains at least two selectable markers, one for the expression cell type and one for the amplification cell type. Any selectable marker known in the art or those described herein may be used provided it functions in the expression system being utilized.

The vectors encompassed by the invention can be obtained using recombinant cloning methods and/or by chemical synthesis. A vast number of recombinant cloning techniques such as PCR, restriction endonuclease digestion and ligation are well known in the art, and need not be described in detail herein. One of skill in the art can also use the sequence data provided herein or that in the public or proprietary databases to obtain a desired vector by any synthetic means available in the art. Additionally, using well-known restriction and ligation techniques, appropriate sequences can be excised from various DNA sources and integrated in operative relationship with the exogenous sequences to be expressed in accordance with embodiments described herein.

### Kits

Also provided are kits for making antigen binding proteins. In some embodiments, the kit comprises two or more of the following components:
1. a first antigen binding fragment comprising a binding motif, such as SpyTag, SpyTag002, SpyTag003, or SnoopTag; and
2. a fusion protein comprising a modified BiCatcher, modified HeteroCatcher, or modified MultiCatcher, the individual elements of which may, optionally, be joined by a linker sequence, optionally, comprise a detectable label (e.g., biotin, HRP, or a fluorophore) and/or which may be, optionally, locked; and/or
3. a second antigen binding fragment comprising a binding motif (such as SpyTag, SpyTag002, SpyTag003, or SnoopTag) that undergoes protein ligation with an unlocked BiCatcher, unlocked HeteroCatcher, or unlocked MultiCatcher; and/or
4. a nucleic acid construct comprising a polynucleotide sequence encoding an antigen binding fragment and/or a fusion protein as defined in 1-3.

A kit user can choose at least two components from 1-3 that, when mixed, will form a covalent bond via protein ligation.

Each of the components of the kit can be provided in liquid form (as a solution) or as a solid (e.g., a powder) that is reconstituted with liquid, e.g., buffer, prior to use. In some embodiments, the kit further comprises instructions for ligating one or more of the binding motif pairs.

Methods of making SpyCatcher, SnoopCatcher, DogCatcher or SilkCatcher analogs A cysteine substitution can be made for any amino acid that is present within a SpyCatcher, SnoopCatcher, DogCatcher or SilkCatcher protein as set forth in any one of SEQ ID NOs: 47-51. In general, one or more amino acid in SEQ ID NOs: 47-51, 56, or 58 (referred to as a “target protein”) are mutated to a cysteine residue by performing cysteine-scanning mutagenesis on the target protein and measuring effects on the ability of the mutated target protein to catalyze protein ligation with an appropriate substrate, such as a protein containing a SpyTag, SnoopTag, DogTag, SilkTag, or analogs thereof. For those mutated proteins that still retain the ability to catalyze protein ligation with an appropriate substrate, these proteins are then locked with a chemical cage, e.g., a disulfide-forming molecule such as Ellmann's reagent (5,5′-dithio-bis-(2-nitrobenzoic acid), DTNB) or HPDP-biotin (N-[6-(biotinamido)hexyl]-3′-(2′-pyridyldithio)propionamide) and the “locked” protein is tested for its ability to catalyze protein ligation with an appropriate substrate. If the locked SpyCatcher, SnoopCatcher, DogCatcher or SilkCatcher analog is unable to catalyze protein ligation, it is considered a suitable analog. The locked analog can then be unlocked and tested for its ability to catalyze protein ligation with an appropriate substrate.

In other embodiments, one or more unnatural amino acid mutations can be introduced into a SpyCatcher, SnoopCatcher, DogCatcher or SilkCatcher protein as set forth in any one of SEQ ID NOs: 47-51. In general, one or more amino acid in SEQ ID NOs: 47-51, 56, or 58 (referred to as a “target protein”) are mutated to an unnatural amino acid, such as unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids. Such mutated target proteins may be locked by way of the introduction of the one or more unnatural amino acids and the effect of the mutation can be measured by the effect the mutation has on the ability of the mutated target protein to catalyze protein ligation with an appropriate substrate, such a SpyTag, SnoopTag, DogTag, or SilkTag or a protein comprising SpyTag, SnoopTag, DogTag, or SilkTag. If the locked SpyCatcher, SnoopCatcher, DogCatcher or SilkCatcher analog is unable to catalyze protein ligation, it is considered a suitable analog. The ability of the mutated target protein to catalyze protein ligation can be confirmed by uncaging the caged amino acid (e.g., using light or chemical uncaging) introduced into the target protein and the ability of the mutated target protein to catalyze protein ligation can be assessed.

### Uses of Antigen Binding Proteins

As discussed above, antigen binding proteins that are made by protein ligation circumvents the genetic engineering steps currently needed to make such binding reagents. Multivalency increases the sensitivity of the antigen binding proteins which is a useful characteristic in such applications as Western blotting, flow cytometry, immunohistochemistry, in some enzyme-linked immunoassays. Bispecificity or multispecificity is useful for increasing target specificity, target affinity or for binding to two or more different antigens simultaneously. Bispecific antigen binding proteins are also of increasing significance as therapeutic drugs. Addition of a second function by protein ligation is useful for preparing labeled antigen binding proteins for applications such as Western blotting, flow cytometry, immunohistochemistry, enzyme-linked immunoassays, for directed immobilization, or for other applications. The antigen binding proteins can be directly labeled for use in various immunoassays or can be indirectly labeled using antibodies specific for the antigen binding proteins provided by this disclosure that are labeled with a detectable label.

Thus, in various embodiments, the disclosed antigen binding proteins can be used in a variety of applications, such as Western blotting, lateral flow immunoassays, singleplex or multiplex immunoassays, flow cytometry, immunohistochemistry, enzyme-linked immunoassays as are known in the art. For example, lateral flow immunoassays can be performed in a manner analogous to those disclosed in U.S. Pat. Nos. 5,851,776 and 6,777,190 (each of which is hereby incorporated by reference in their entireties and which relate to lateral flow chromatographic assays on a membrane or other porous or non-porous materials). Other methods in which the disclosed antigen binding proteins can be used included, and are not limited to flow cytometry techniques, immunodiffusion techniques, immunoelectrophoretic techniques, light scattering immunoassays, agglutination techniques, labeled immunoassays such as radiolabeled immunoassays, and enzyme-based immunoassays such as colorimetric assays, chemiluminescence, preferably electrochemiluminescence, immunoassays and immunofluorescence techniques. The person skilled in the art is familiar with these methods, which are also described in the state of the art, for example in Zane, H. D. (2001): Immunology—Theoretical & Practical Concepts in Laboratory Medicine, W. B. Saunders Company, in particular in Chapter 14.

In other embodiments, the disclosed antigen binding proteins can be used to target a specific antigen on a cell surface to direct cytotoxic agents to the cell in both in vivo and in vitro settings. Thus, the antigen binding fragments can be conjugated to a therapeutic agent, such as a cytotoxin or a radioactive metal ion or radioisotope. Examples of radioisotopes include, but are not limited to, I-131, 1-123, 1-125, Y-90, Re-188, Re-186, At-211, Cu-67, Bi-212, Bi-213, Pd-109, Tc-99, In-111, and the like. In some embodiments, the therapeutic agent can be a toxin such as abrin, ricin A, Pseudomonas exotoxin, or diphtheria toxin or another therapeutic agent, such as ozogamicin, vedotin, emtansine, deruxtecan, or govitecan. Techniques for conjugating such therapeutic moieties to antibodies are well known.

### Additional Disclosure and Claimable Subject Matter

1. A SpyCatcher analog comprising:
a) a SpyCatcher analog comprising any one of SEQ ID NOs: 1-4 or 7-46 or a mutated SpyCatcher analog having at least 60% sequence identity to said SpyCatcher analog, with the proviso that said mutated SpyCatcher analog having 60% sequence identity to said SpyCatcher analog catalyzes protein ligation and the cysteine residue introduced into said SpyCatcher analog is not substituted in said mutated SpyCatcher analog;
b) a SpyCatcher analog comprising one or more of the mutations in the SpyCatcher protein of SEQ ID NO: 47, said one or more mutations being selected from the group consisting of I27C, F29C, A42C, M44C, W57C, S59C, F75C, Y84C, A87C, F92C, and V94C, or a mutated SpyCatcher analog having at least 60% sequence identity to said SpyCatcher analog, with the proviso that said SpyCatcher analog or said mutated SpyCatcher analog catalyzes protein ligation and the cysteine residue(s) introduced into said SpyCatcher analog or said mutated SpyCatcher analog is/are not substituted;
c) a SpyCatcher analog comprising one or more of the mutations in the SpyCatcher protein of SEQ ID NO: 48, said one or more mutations being selected from the group consisting of I27C, F29C, A42C, M44C, W57C, S59C, F75C, Y84C, A87C, F92C, and V94C, or a mutated SpyCatcher analog having at least 60% sequence identity to said SpyCatcher analog, with the proviso that said SpyCatcher analog or said mutated SpyCatcher analog catalyzes protein ligation and the cysteine residue(s) introduced into said SpyCatcher analog or said mutated SpyCatcher analog is/are not substituted in said SpyCatcher analog or said polypeptide;
d) a SpyCatcher analog comprising one or more of the mutations in the SpyCatcher protein of SEQ ID NO: 49, said one or more mutations being selected from the group consisting of I27C, F29C, A42C, M44C, W57C, S59C, F75C, Y84C, A87C, F92C, and V94C, or a mutated SpyCatcher analog having at least 60% sequence identity to said SpyCatcher analog, with the proviso that said SpyCatcher analog or said mutated SpyCatcher analog catalyzes protein ligation and the cysteine residue(s) introduced into said SpyCatcher analog or said mutated SpyCatcher analog is/are not substituted in said SpyCatcher analog or said polypeptide;
e) a SpyCatcher analog comprising one or more of the mutations in the SpyCatcher protein of SEQ ID NO: 50, said one or more mutations being selected from the group consisting of I6C, F8C, A21C, M23C, W36C, S38C, F54C, Y63C, A66C, F71C, and V73C, or a mutated SpyCatcher analog having at least 60% sequence identity to said SpyCatcher analog, with the proviso that said SpyCatcher analog or said mutated SpyCatcher analog catalyzes protein ligation and the cysteine residue(s) introduced into said SpyCatcher analog or said mutated SpyCatcher analog is/are not substituted in said SpyCatcher analog or said polypeptide;
f) a SpyCatcher analog comprising one or more of the mutations in the SpyCatcher protein of SEQ ID NO: 47, said one or more mutations being selected from the group consisting of 127, F29, A42, M44, W57, S59, F75, Y84, A87, F92, and V94, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, wherein Lys31 and Glu77 are not substituted in said SpyCatcher analog or mutated SpyCatcher analog and the mutations introduced to form said SpyCatcher analog are not changed in the mutated SpyCatcher analog and the one or more mutations that are introduced to form the SpyCatcher analog of SEQ ID NO: 47 are: a) unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids; or b) cysteine residues in combination with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids,
   wherein a SpyCatcher analog or mutated SpyCatcher analog containing one or more caged unnatural amino acids has the ability to catalyze protein ligation after uncaging of said one or more caged unnatural amino acids;
g) a SpyCatcher analog comprising one or more of the mutations in the SpyCatcher protein of SEQ ID NO: 48, said one or more mutations being selected from the group consisting of 127, F29, A42, M44, W57, S59, F75, Y84, A87, F92, and V94, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, with the proviso that said SpyCatcher analog or said mutated SpyCatcher analog catalyzes protein ligation, wherein Lys31 and Glu77 are not substituted in said SpyCatcher analog or mutated SpyCatcher analog and the mutations introduced to form said SpyCatcher analog are not changed in the mutated SpyCatcher analog and the one or more mutations that are introduced to form the SpyCatcher analog of SEQ ID NO: 48 are: a) unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids; or b) cysteine residues in combination with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids, wherein a SpyCatcher analog or mutated SpyCatcher analog containing one or more caged unnatural amino acids has the ability to catalyze protein ligation after uncaging of said one or more caged unnatural amino acids;
h) a SpyCatcher analog comprising one or more of the mutations in the SpyCatcher protein of SEQ ID NO: 49, said one or more mutations being selected from the group consisting of 127, F29, A42, M44, W57, S59, F75, Y84, A87, F92, and V94, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, wherein Lys31 and Glu77 are not substituted in said SpyCatcher analog or mutated SpyCatcher analog and the mutations introduced to form said SpyCatcher analog are not changed in the mutated SpyCatcher analog and the one or more mutations that are introduced to form the SpyCatcher analog of SEQ ID NO: 49 are: a) unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids; or b) cysteine residues in combination with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids, wherein a SpyCatcher analog or mutated SpyCatcher analog containing one or more caged unnatural amino acids has the ability to catalyze protein ligation after uncaging of said one or more caged unnatural amino acids;
i) a SpyCatcher analog comprising one or more of the mutations in the SpyCatcher protein of SEQ ID NO: 50, said one or more mutations being selected from the group consisting of 16, F8, A21, M23, W36, S38, F54, Y63, A66, F71, and V73, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, wherein Lys31 and Glu77 are not substituted in said SpyCatcher analog or mutated SpyCatcher analog and the mutations introduced to form said SpyCatcher analog are not changed in the mutated SpyCatcher analog and the one or more mutations that are introduced to form the SpyCatcher analog of SEQ ID NO: 50 are: a) unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids; or b) cysteine residues in combination with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids;
   wherein a SpyCatcher analog or mutated SpyCatcher analog containing one or more caged unnatural amino acids has the ability to catalyze protein ligation after uncaging of said one or more caged unnatural amino acids; or
j) a SpyCatcher, SpyCatcher002, SpyCatcher003, or SpyCatcher short analog in which the reactive lysine (at position 31 of SpyCatcher, SpyCatcher002, or SpyCatcher003 or position 10 of SpyCatcher short) is “locked” with a photocaged lysine, such as (S)-2-Amino-6-((((7-hydroxy-2-oxo-2H-chromen-4-yl)methoxy)carbonyl)amino)hexanoic acid or a chemically caged lysine such as N6-(((4-azidobenzyl)oxy)carbonyl)-L-lysine).

2. A fusion protein comprising at least one SpyCatcher analog according to claim 1 and at least one non-mutated protein selected from the group consisting of SEQ ID NO: 47, 48, 49, 50, 51, 56, or 58 and/or at least one a mutated protein having at least 60% sequence identity to SEQ ID NO: 47, 48, 49, 50, 51, 56, or 58, with the proviso that:
a) said mutated protein having 60% sequence identity to SEQ ID NO: 47, 48, 49, 50, 51, 56, or 58 catalyzes protein ligation.

3. The fusion protein according to embodiment 2, wherein the binding motifs are joined to one another via a linker.

4. The fusion protein according to embodiment 3, wherein the linker is a heterobifunctional chemical linker or is a peptide linker.

5. The fusion protein according to any one of embodiments 2-4, wherein the linker is a peptide linker, such as -(GGGS)n-, where n is 1, 2, 3, 4, or 5, (Gly)n, where n is an integer between 2 and 50, Gly-Ser, Gly-Ser-Gly-Ser, Ala-Ser, Gly-Gly-Gly-Ser, Gly₄-Ser, (Gly₄-Ser)₂, (Gly₄-Ser)₃, (Gly₄-Ser)₄, (Gly₄-Ser)₂-Gly-Ala-Gly-Ser-Gly₄-Ser, Gly-(Gly₄-Ser)₂, Gly₄-Ser-Gly, Gly-Ser-Gly₂, Gly-Ser-Gly₂-Ser, ENLYFQG (SEQ ID NO: 61), GSENLYFQGSG (SEQ ID NO: 62), Ala-(Glu-Ala-Ala-Ala-Lys)n-Ala (n=1-5) or GAP (Gly Ala Pro) sequence and the peptide linker may be the same or different between elements of the fusion protein.

6. An antigen binding protein comprising:
one or more first antigen binding fragments comprising a binding motif comprising a SpyTag analog or any one of SEQ ID NOs: 52-55 and, optionally, one or more second antigen binding fragments comprising a binding motif comprising a SpyTag analog or any one of SEQ ID NOs: 52-55 and a fusion protein comprising a SpyCatcher analog according to embodiment 2
wherein the binding motifs are covalently conjugated to the fusion protein via protein ligation, provided that:
a) if one of the elements of the fusion protein is SEQ ID NO: 51, then one of the binding motifs of the antigen binding fragments is SEQ ID NO: 55 or a sequence having at least 60% identity to SEQ ID NO: 55;
b) if one of the elements of the fusion protein is SEQ ID NO: 56, then one of the binding motifs of the antigen binding fragments is SEQ ID NO: 57 or a sequence having at least 60% identity to SEQ ID NO: 57; and/or
c) if one of the elements of the fusion protein is SEQ ID NO: 58, then one of the binding motifs of the antigen binding fragments is SEQ ID NO: 59 or a sequence having at least 60% identity to SEQ ID NO: 59.

7. The antigen binding protein according to embodiment 6, wherein the antigen binding protein is monospecific and dimeric, monospecific and multimeric, bispecific, bispecific and dimeric, or bispecific and multimeric, or multispecific and multimeric.

8. The antigen binding protein according to embodiment 6, wherein the binding motifs are located at a C terminus, an N-terminus or embedded within an amino acid sequence of the antigen binding fragments.

9. The antigen binding protein according to embodiment 6, wherein the fusion protein or polypeptide further comprises a detectable label.

10. The antigen binding protein according to embodiment 9, wherein the detectable label is a fluorophore, a fluorescent protein, a radioisotope, digoxygenin, biotin, or an enzyme.

11. A solid support comprising a SpyCatcher analog according to embodiment 1 or a fusion protein according to any one of embodiments 2-5.

12. The solid support according to embodiment 11, wherein the SpyCatcher analog or the SpyCatcher analog of the fusion protein is locked with a disulfide forming reagent, such as Npys-Cys-StrepTag2 or other Npys-Cys-peptides, Ellmann's reagent (5,5′-dithio-bis-(2-nitrobenzoic acid), DTNB) or HPDP-biotin (N-[6-(biotinamido)hexyl]-3′-(2′-pyridyldithio)propionamide) or other disulfide forming reagents or other chemical reagents which can be selectively removed, e.g., by photo- or chemical uncaging, or by introduction of unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids.

13. A SpyLock protein comprising a SpyCatcher analog according to embodiment 1, said SpyLock protein being locked with: a) a reagent that forms a disulfide bond (a disulfide forming reagent) with the cysteine that has been introduced into said SpyCatcher analog; b) by formation of intra-chain disulfide bonds between cysteine residues introduced into said SpyCatcher analog; or c) with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids.

14. The SpyLock protein according to embodiment 13, wherein said SpyLock protein has been locked with an unnatural amino acid, or by formation of a disulfide bond by reaction of one or more introduced cysteines with Npys-Cys-StrepTag2, Npys-Cys-His6, Npys-Cys-Flag, Npys-Cys-myc, Ellmann's reagent (5,5′-dithio-bis-(2-nitrobenzoic acid), DTNB) or HPDP-biotin (N-[6-(biotinamido)hexyl]-3′-(2′-pyridyldithio)propionamide) or other disulfide forming reagents.

15. A fusion protein according to any one of embodiments 2-5, said fusion protein having been locked with: a) a reagent that forms a disulfide bond with the cysteine that has been introduced into said SpyCatcher analog; b) by formation of intra-chain disulfide bonds between cysteine residues introduced into said SpyCatcher analog; or c) with an unnatural amino acid that is caged, such as with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids.

16. The fusion protein according to embodiment 15, wherein said fusion protein has been locked with an unnatural amino acid (such as with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids), or by formation of a disulfide bond by reaction of one or more introduced cysteines with Npys-Cys-StrepTag2, Npys-Cys-His6, Npys-Cys-Flag, Npys-Cys-myc, Npys-Cys-peptide, Ellmann's reagent (5,5′-dithio-bis-(2-nitrobenzoic acid), DTNB) or HPDP-biotin (N-[6-(biotinamido)hexyl]-3′-(2′-pyridyldithio)propionamide) or other disulfide forming reagents.

17. A polynucleotide sequence encoding a SpyCatcher analog according to embodiment 1 or a fusion protein comprising said SpyCatcher analog.

18. A nucleic acid construct comprising the polynucleotide according to embodiment 17.

19. A vector comprising a polynucleotide according to embodiment 18 or a nucleic acid construct comprising said polynucleotide.

20. A host cell comprising a polynucleotide according to embodiment 18, a nucleic acid construct comprising said polynucleotide, or a vector comprising said polynucleotide or said nucleic acid construct.

21. A method for identifying lockable SpyCatcher, SnoopCatcher, DogCatcher, or SilkCatcher protein comprising:
a) introducing one or more amino acid mutation(s) into a protein comprising any one of SEQ ID NOs: 47-51, 56, or 58 or a protein having at least 60% sequence identity to any one of SEQ ID NO: 47-51, 56, or 58 and catalyzing protein ligation, said one or more amino acid mutation(s) comprising:
   i) the introduction of one or more cysteine residue as said one or more amino acid mutations; or
   ii) introduction of one or more unnatural amino acid selected from a photocaged cysteine, photocaged lysine, photocaged tyrosine, chemically caged lysines, or combinations of these unnatural amino acids, wherein the introduction of the one or more unnatural amino acid forms a locked mutated protein; or
   iii) the introduction of one or more cysteine residues and one or more unnatural amino acids selected from with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids;
b) contacting a protein mutated to contain one or more cysteine residue with a locking agent, such as Ellmann's reagent (5,5′-dithio-bis-(2-nitrobenzoic acid), DTNB) or HPDP-biotin (N-[6-(biotinamido)hexyl]-3′-(2′-pyridyldithio)propionamide), that forms a disulfide bond with the cysteine introduced into said mutated protein and forming a disulfide bond between said cysteine and said locking agent to form a potentially locked mutated protein or forming intra-chain disulfide bonds between two cysteine residues that have been introduced into said mutated protein to form a potentially locked mutated protein; and
c) contacting said potentially locked mutated protein with a substrate comprising a binding motif specific for said potentially locked mutated protein and determining if said potentially locked mutated protein has catalyzed peptide bond formation between said potentially locked mutated protein and said substrate comprising a binding motif specific for said locked mutated protein, wherein the lack of a peptide bond between said potentially locked mutated protein and said substrate comprising a binding motif specific for said potentially locked mutated protein identifies the potentially locked mutated protein as a lockable protein, while the formation of a peptide bond between said substrate comprising a binding motif specific for said potentially locked mutated protein and said potentially locked mutated protein identifies the mutation in said lockable protein as unable to lock the protein.

22. The method according to embodiment 21, wherein:
a) the protein into which one or more amino acid mutation is introduced is selected from the group consisting of SpyCatcher, SpyCatcher short, SpyCatcher002, SpyCatcher003, SnoopCatcher, DogCatcher, SilkCatcher; and
b) the substrate is selected from the group consisting of SpyTag, SpyTag002, SpyTag003, SnoopTag, DogTag, and SilkTag or analogs thereof that are, optionally, labeled.

23. The method according to embodiments 21-22, wherein determining if said locked mutated protein has catalyzed a peptide bond formation between said locked mutated protein and said substrate comprising a binding motif specific for said locked mutated protein comprises detecting a signal from a labeled substrate.

24. The method according to embodiments 21-23, wherein the substrate is labeled with a fluorophore, a fluorescent protein, biotin, an enzyme, luciferase, a split fluorescent protein, a split luciferase, a radioisotope, or enzymes.

25. The method according to any one of embodiments 21-23, wherein said detection comprises detecting a change of electrophoretic mobility of said mutated protein, the absence of a change in electrophoretic mobility indicating that said amino acid mutation identifies the potentially locked mutated protein as a lockable protein.

26. A lockable analog of SpyCatcher, SpyCatcher002, SpyCatcher003, SnoopCatcher, DogCatcher, or SilkCatcher produced by the method of any one of embodiments 21-25.

27. The lockable analog according to embodiment 26, said lockable analog has been locked with: locked with: a) a reagent that forms a disulfide bond (a disulfide forming reagent) with the cysteine that has been introduced into said analog; b) by formation of intra-chain disulfide bonds between cysteine residues introduced into said analog; or c) with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids.

28. A lockable SpyCatcher, SpyCatcher002, SpyCatcher003, SpyLock protein, fusion protein, or antigen binding protein of any of the preceding embodiments, wherein the unnatural amino acid is, one or more: photocaged cysteine, photocaged lysine, photocaged tyrosine, chemically caged lysine, or a combination thereof.

### Examples

The following examples are provided by way of illustration only and not by way of limitation. Those of skill in the art will readily recognize a variety of non-critical parameters that could be changed or modified to yield essentially the same or similar results.

### 1.1 Generation of Mutated SpyCatchers

We designed SpyCatchers in which Serine 61 (Serine 59 in SpyCatcher 3) was mutated to cysteine.

The DNA was cloned into pET-28a(+) vector and transformed into E. coli BL21. For the expression, an 2×YT medium was inoculated with this clone and grown for 18-24 h at 37° C. 1 ml of this preculture was used to inoculated 250 ml 2×YT expression culture. After 1 h of shaking at 30° C. expression was induced with 1 mM IPTG. After overnight shaking at 30° C., E. coli were pelleted by centrifugation and lysed with 5 ml lysis buffer. The SpyCatcher was purified via IMAC, rebuffered to PBS and sterile filtered. Purity was estimated by SDS gel electrophoresis and was >90% for all three constructs (SEQ ID NOs: 1-3).

### 1.2 Inhibiting Modification of Mutated SpyCatcher

42.5 μM reduced, modified SpyCatchers (SEQ ID NOs: 1-3) in PBS pH 7.4 with 1 mM EDTA were incubated with 425 μM HPDP-biotin (Thermo Scientific) for 18 hours followed by buffer exchange into PBS. Alternatively, 50 μM reduced, modified SpyCatchers (Seq ID NOs: 1-3) in PBS pH 7.4 with 1 mM EDTA were incubated with 500 μM Ellmann's reagent (5,5′-Dithio-bis-(2-nitrobenzoic acid), Thermo Scientific) for 30 minutes followed by buffer exchange into PBS. These reactions create a disulfide bond between the introduced cysteines and the labeling reagent to “lock” the modified SpyCatcher proteins.

1.3 Testing SpyTag Coupling Activity of Mutated SpyCatchers with and without Inhibiting Modification

10 μM modified SpyCatchers with a HPDP-biotin label were incubated with 20 μM MBP-SpyTag002 with or without 5 mM TCEP. Samples were taken after 0 min, 5 min, 10 min, 30 min, 60 min, 18 hours and the reaction was stopped by boiling with SDS PAGE buffer. Samples were analyzed by SDS PAGE (FIGS. 3, 4, and 5).

The reaction with SpyTag is significantly inhibited in the HPDP-biotin labeled Catchers in absence of the reducing agent TCEP, whereas removal of biotin by reduction of the disulfide bond between biotin and cysteine leads to strong SpyTag-SpyCatcher reactivity, with the reaction being complete within one hour.

### 2. Bispecific Antibodies

### 2.1 Generation of SpyCatcher-Mutated SpyCatcher Fusion Proteins (Mutated BiCatcher)

We designed and expressed two versions of SpyCatcher003-mutated SpyCatcher003 fusion proteins. The first version is SpyCatcher003 with S59C mutation is at the N-terminus followed by the original (unmutated) SpyCatcher003 (SEQ ID NO: 5) and, in the second version, the mutated SpyCatcher003 is at the C-terminus and the original (unmutated) SpyCatcher003 is at the N-terminus (SEQ ID NO: 6). The DNA was cloned into pET-28a(+) vector and transformed into E. coli BL21. Expression and purification was done as described in example 1.1. Purity was estimated by SDS gel electrophoresis and was >90% for both constructs.

### 2.2 Inhibition of Mutated BiCatcher and Optimization of the First Coupling Reaction

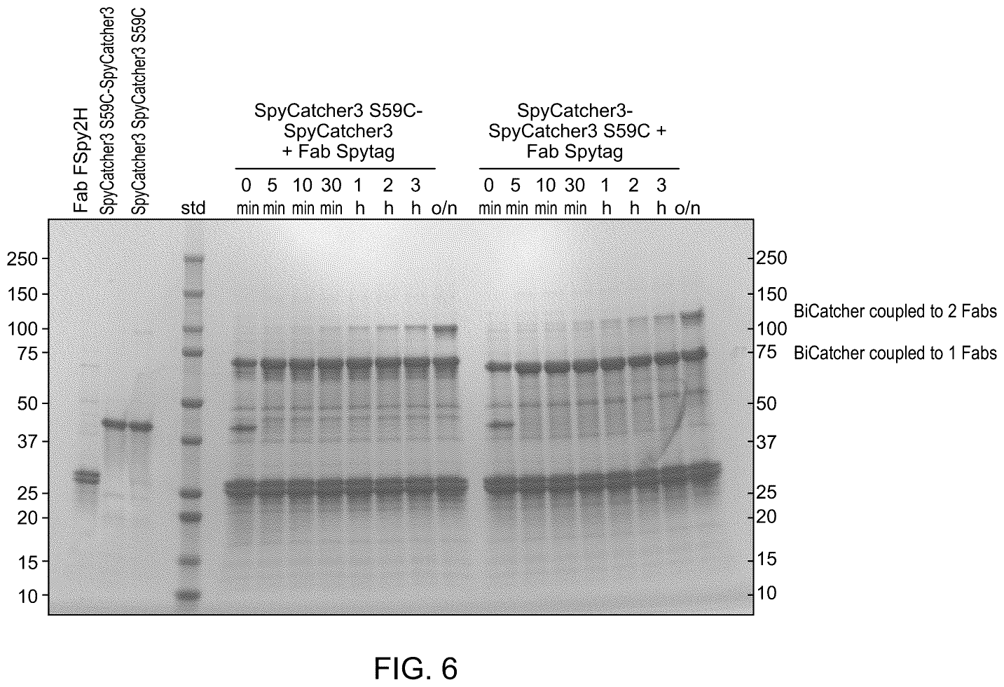

BiCatchers (SEQ ID NOs: 5 and 6) were labeled with HPDP-biotin as described above in example 1.2. 6 μM labeled BiCatchers were incubated with 15 μM Fab-Flag-SpyTag002-His6 in PBS. Samples were taken after 0 min, 5 min, 10 min, 30 min, 1 h, 2 h, 3 h, and 18 hours and the reaction was stopped by boiling with SDS PAGE buffer. Samples were analyzed by SDS PAGE (FIG. 6).

Treatment of these mutated BiCatchers with HPDP-biotin resulted in proteins, in which the regular SpyCatcher was accessible, while the mutated SpyCatcher was prevented from reacting on the time scale of 1 hour (FIG. 6). We also saw a slight advantage in the degree of inhibition for SEQ ID NO: 6.

### 2.3 Unlocking the Mutated BiCatcher and Coupling Reaction

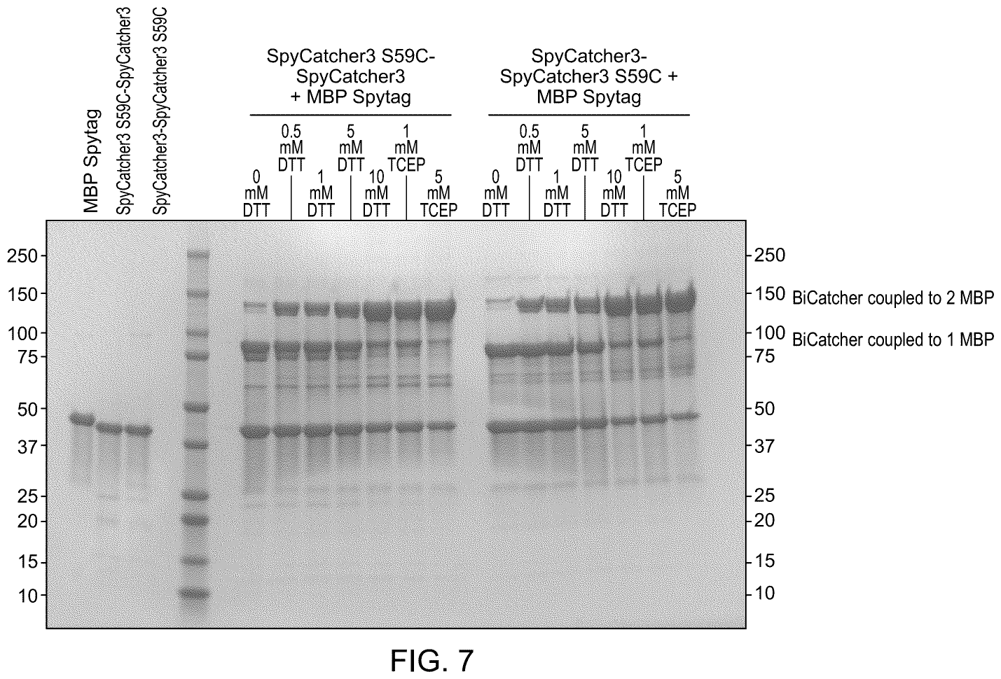

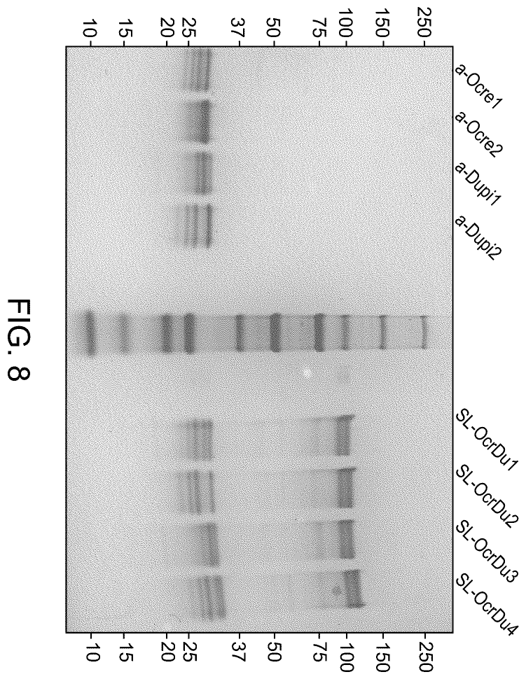

8 μM HPDP-biotin labeled BiCatchers and 20 μM MBP-SpyTag002 were incubated with varying concentration of reducing agents (TCEP or DTT) for 1 hour at room temperature, followed by SDS PAGE (FIG. 7). While coupling without reducing agent yields mainly mono-coupled product, DTT or TCEP addition leads to BiCatcher coupled to two MBP molecules. We found that 5 mM TCEP is sufficient for removing the block by reduction of the disulfide bond at cysteine 59.

Since the inhibition of the mutated SpyCatcher was slightly more pronounced in the variant with the modified Catcher at the C-terminus (SEQ ID NO: 6), we decided to use this protein for further experiments.

2.4 Bispecific Antibodies were Constructed in the Following Fashion

1 μM of SpyCatcher003-SpyCatcher003-S59C-biotin were incubated with 1.25 μM of a first SpyTagged antibody for 20 minutes. The reaction mixture was incubated with streptavidin Dynabeads (Thermo Scientific) for 30 minutes. Beads were washed 3× with PBS/0.05% Tween (PBST) to remove uncoupled antibody and then incubated with 5 mM TCEP for 1 hour to release the BiCatcher-Antibody1 from the beads while simultaneously reactivating the reactivity of the SpyCatcher003 S59C domain. Assuming complete binding and release of the BiCatcher, 1.25 equivalents of a second antibody were added and reacted for one hour to form bispecific antibodies. First antibodies in this example were anti-idiotypic antibodies against ocrelizumab (AbD50987ad and AbD50118ad, a-Ocre 1&2); second antibodies were anti-idiotypic antibodies against dupilumab (AbD50196ad and AbD52403ad, a-Dupi 1&2). All 4 combinations of these antibodies were generated. Coupling reactions were analyzed by SDS PAGE (FIG. 8). Additionally, homodimers were generated with the same procedure as negative controls.

### 2.5 Inactivation of the Reducing Agent

One potential caveat of the reversibly inhibitable BiCatcher approach is the presence of reducing agent in the final bispecific product, as the internal disulfide bonds within the antibodies could become reduced over time as well, causing the antibodies to lose their folding and thereby antigen binding capacity. It would of course be possible to purify the final product with size exclusion chromatography, but this would add a laborious step, dilute the sample, and would require an additional measurement of the final protein concentration. Instead, we decided to test inactivation of the reducing agent TCEP with azide-PEG4-azide, as described 15 in the literature (Kantner et al., 2017). Briefly, 5 mM TCEP in PBS were incubated with 10×, 20×, and 40× molar excesses of azide-PEG-azide for one hour. Remaining TCEP was quantified by mixing with an equal volume of 80 μg/ml Ellmann's reagent, incubating for 15 minutes, measuring absorption at 412 nm, and comparing the result to a standard curve of known TCEP concentrations.

Indeed, we could confirm that a tenfold excess of azide-PEG4-azide removed reducing activity of TCEP almost quantitatively within one hour (Table 1). Conveniently, this reaction can be performed in parallel with the coupling of the second antibody.

TABLE 1
Remaining TCEP (from a starting concentration of 5 mM)
after one hour of incubation with azide-PEG4-azide

| | residual | % quenched |
|---|---|---|
| | TCEP [μM] | TCEP |
| TCEP/10× azide- | 133 | 97.3 |
| PEG-azide | | |
| TCEP/20× azide- | 32 | 99.4 |
| PEG-azide | | |
| TCEP/40× azide- | 19 | 99.6 |
| PEG-azide | | |

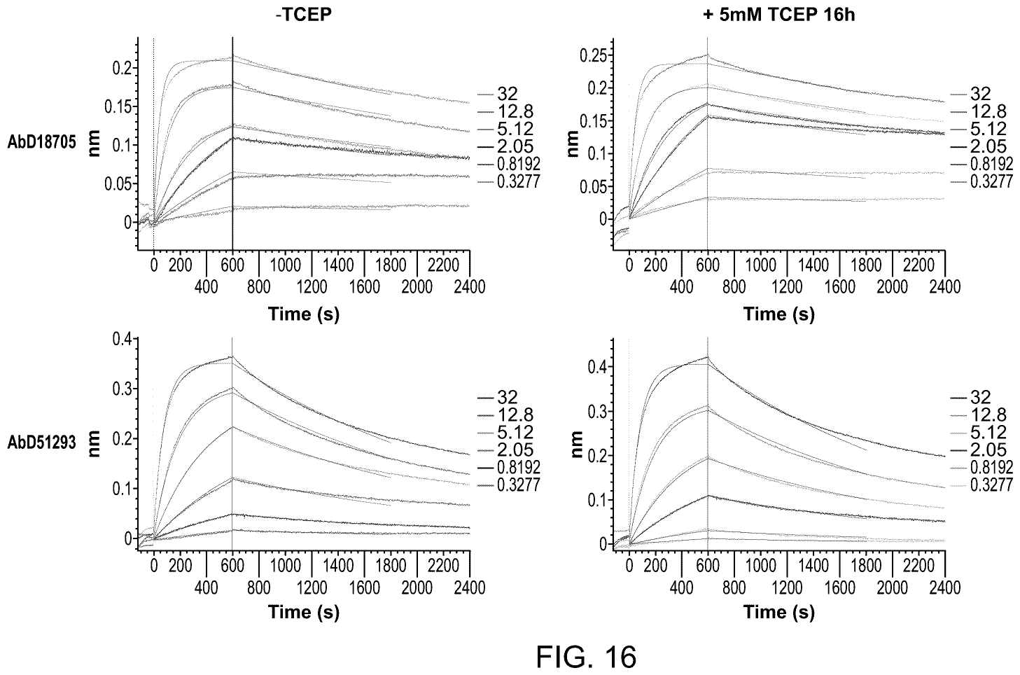

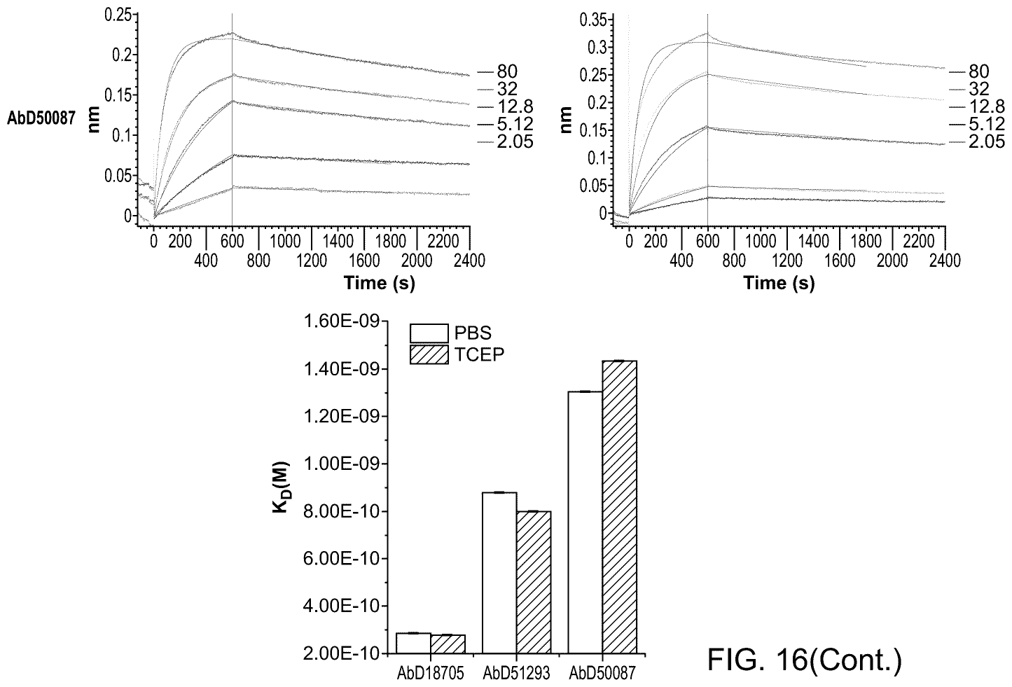

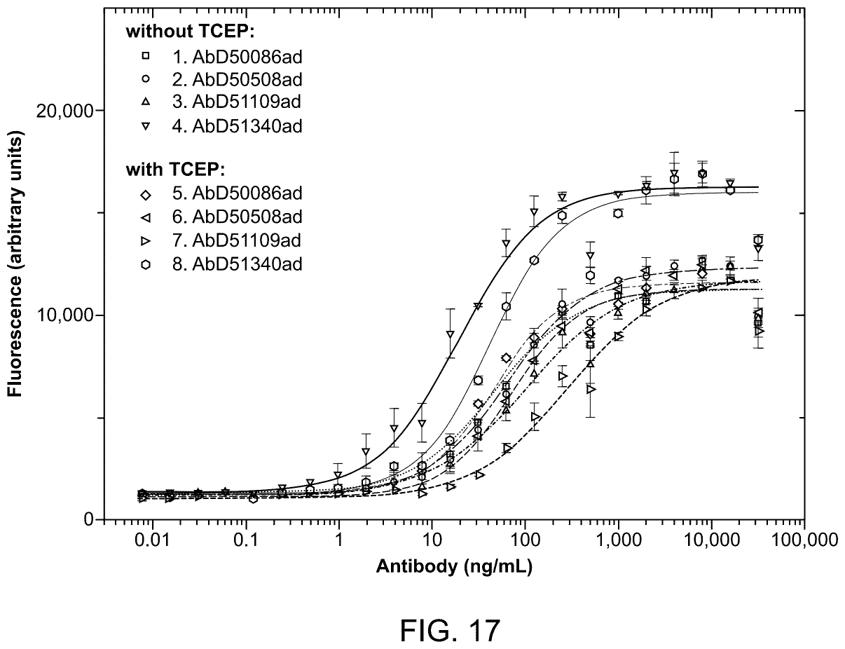

As discussed above, using TCEP in the presence of antibodies raises the concern that the intradomain disulfide bonds of the antibodies are also reduced, and the antibodies thereby lose their function. The inter-chain disulfide between heavy and light chains is not required for the Fab stability and is not present in SpyTagged Fab fragments used in these examples. To quantify the impact of TCEP, we incubated three Fab fragments against GFP with 5 mM TCEP for 16 hours, the longest time period Fabs would be exposed to TCEP during the generation of bispecific antibodies. This did not change their affinity to GFP as measured by biolayer interferometry (BLI) (FIG. 16, Table 2). Even incubating Fab fragments with 5 mM TCEP for an entire week mostly had only minor effects on antigen binding ability as measured by titration ELISA (FIG. 17). Nevertheless, even though the amount of TCEP present in the samples would therefore not be problematic for most applications, TCEP can still be removed by quenching with the non-toxic bis-PEG₃-azide, as described above. This procedure lowers the amount of measurable free TCEP by 97.3%, as determined by use of Ellman's reagent.

TABLE 2
Anti-GFP Fab affinites without or
with TCEP incubation for 16 h

| | | | K_{D} in PBS + | |
|---|---|---|---|---|
| | K_{D} in PBS | Fit | 5 mM TCEP | |
| Fab | (M) | Error | (16 h) (M) | Fit Error |
| AbD18707 | 2.9E−10 | 7.9E−13 | 2.8E−10 | 6.9E−13 |
| AbD51293 | 8.8E−10 | 1.8E−12 | 8.0E−10 | 3.6E−12 |
| AbD50087 | 1.3E−09 | 1.7E−12 | 1.4E−09 | 2.3E−12 |

### 2.6 Characterization of the Bispecific Antibody

To demonstrate the successful generation of bispecific antibodies, we performed sandwich ELISAs. In these assays, one antigen (i.e. the therapeutic antibody) is coated, then the bispecific antibody is bound, and detection is performed with an HRP labeled second antigen (therapeutic antibody). The assay is carried out in both possible orientations, i.e. Antigen 1 coated and detection with Antigen 2-HRP or vice versa.

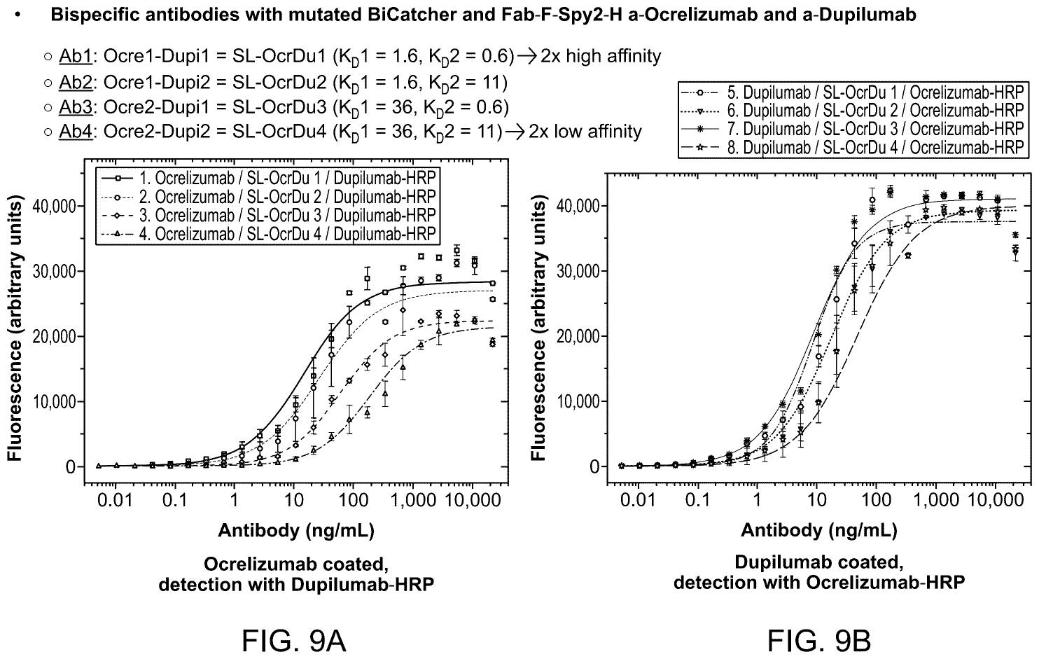

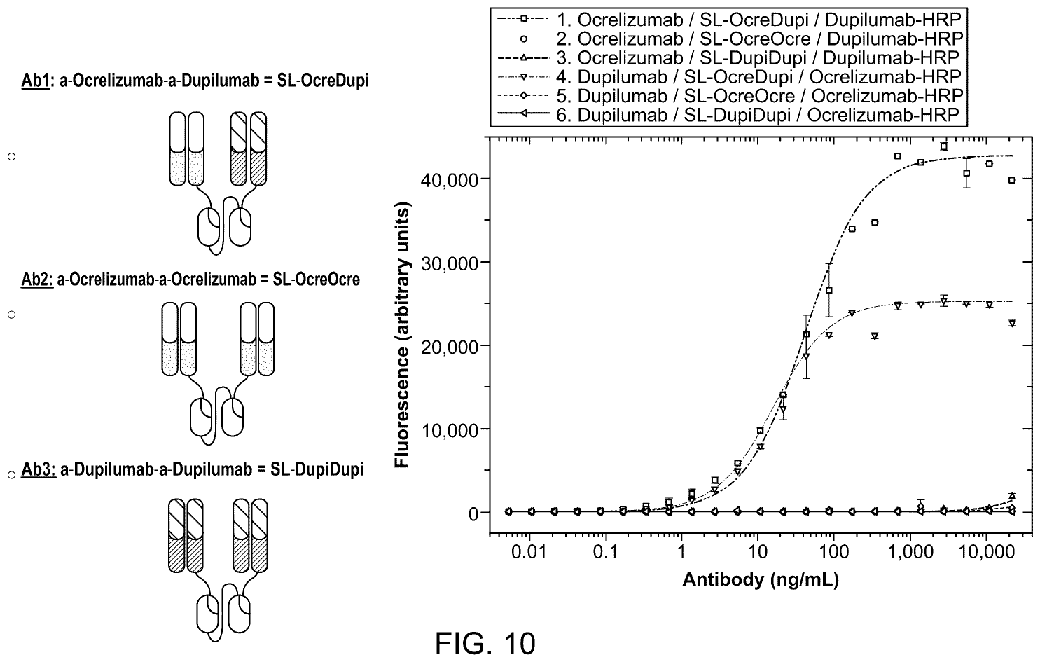

For the ELISA, a 384 well Maxisorp plate (Thermo Scientific, 10395991) was coated with 20 μl ocrelizumab or dupilumab at 1 μg/ml in PBS overnight at 4° C. After washing with PBST the plate was blocked with 100 μl 5% BSA in PBST for 1-2 hours. The plate was washed and a serial dilution of the bispecific antibodies in PBST was added and incubated for 1 hour at room temperature. Detection was performed with HRP conjugated ocrelizumab or dupilumab at 2 μg/ml in HISPEC assay diluent (Bio-Rad Antibodies, BUF049) for 1 hour followed by addition of QuantaBlu fluorescence detection reagent (ThermoScientific, 15169). In all cases tested, bispecificity could be detected in both assay orientations (FIG. 9). In FIG. 10 the assay was repeated with the monospecific homodimers as negative controls.

Thus, the final protocol for generation of bispecific antibody constructs consists of the following steps:
1) Coupling of Biotin-protected mutated SpyCatcher-SpyCatcher with Antibody #1 (10-20 min);
2) Binding of intermediate product to streptavidin beads and washing (15 min);
3) Simultaneous deprotection of mutated SpyCatcher and release of intermediate from beads (1 h);
4) Coupling of Antibody #2 and quenching of reducing agent (1 h).
   The binding of the intermediate to beads is optional, but allows the use of excess antibody #1 in the first coupling step and complete removal of the excess.

For high-throughput screening purposes, however, it might be advantageous to avoid purification of the intermediate. This can be achieved, albeit with a loss of purity, by matching the concentrations of the first coupling between Fab and free Catcher in a 1:1 ratio. This protocol requires less steps which makes it faster and reduces the risk of mix-up errors.

Excess antibody #2 could be removed easily using a solid phase with immobilized SpyCatcher if so desired. Free SpyTagged antibody #2 is coupled to the SpyCatcher resin while the bispecific products flow through the column.

### 3. SpyCatcher Cysteine Mutation Experiments

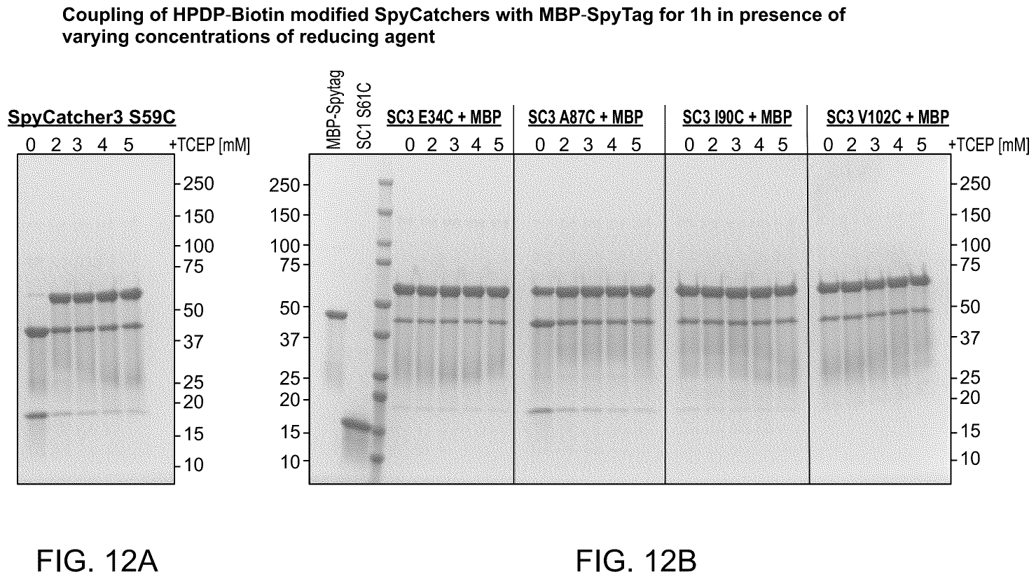

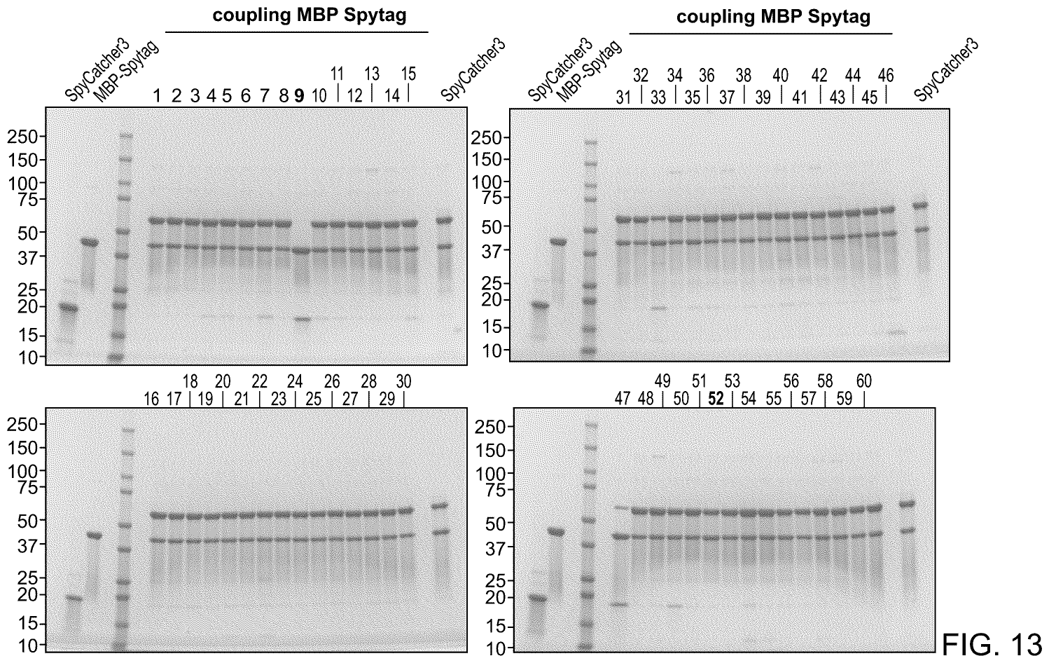

We introduced a Ser59 to Cys mutation into SpyCatcher003 and observed that when labeled with biotin-HPDP, the SpyCatcher did not react anymore with MBP-SpyTag. This inhibition could be reversed by incubation with the reducing agent TCEP (FIG. 12A). We also introduced additional modifications into SpyCatcher in an attempt to target amino acids whose side chains pointed into the binding groove for SpyTag. These mutations were Glu34→Cys, Ala87→Cys, Iso90→Cys, and Val102→Cys. None of these mutations, despite being non-conservative, affected coupling of the Catchers. Once modified with biotin-HPDP, they still coupled with SpyTag (FIG. 12B). There was a mild effect with A87C (about 50% loss of activity without TCEP within one hour reaction time).

### 4. Cysteine Scanning of Remaining Residues in the Structured Region of SpyCatcher

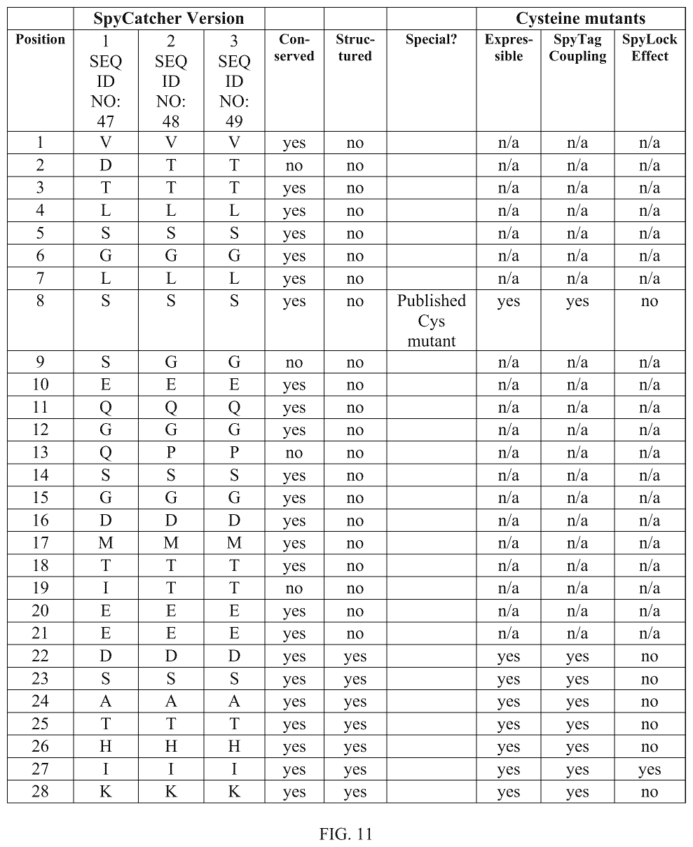

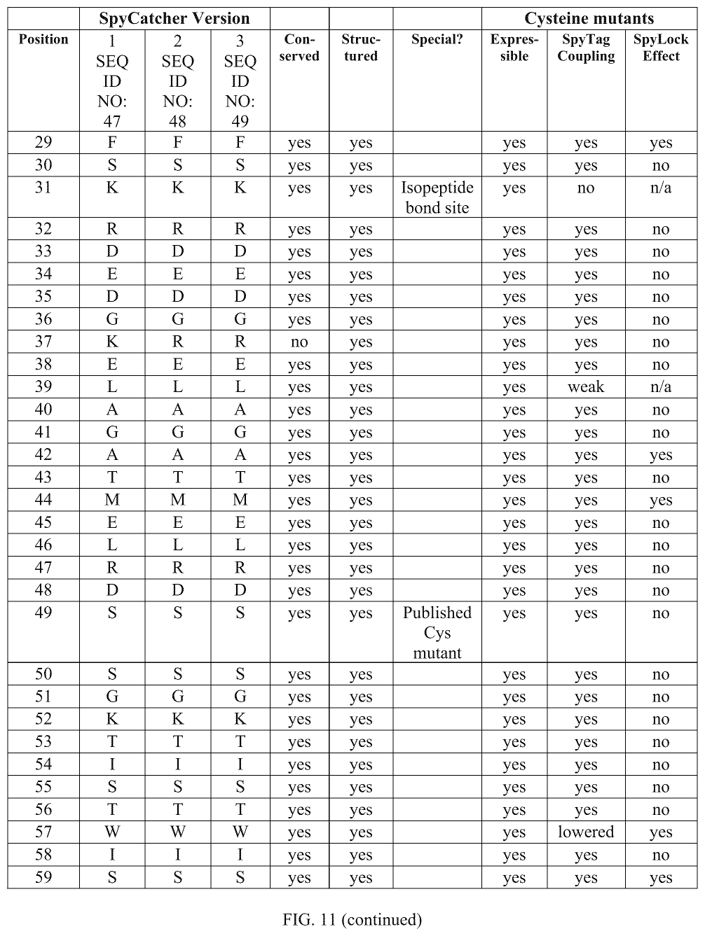

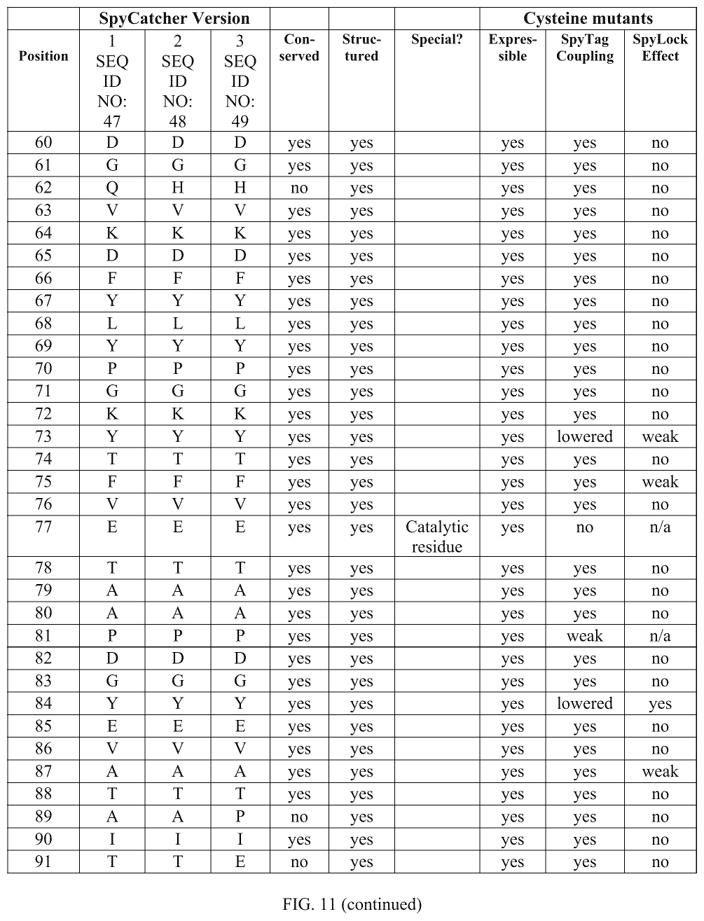

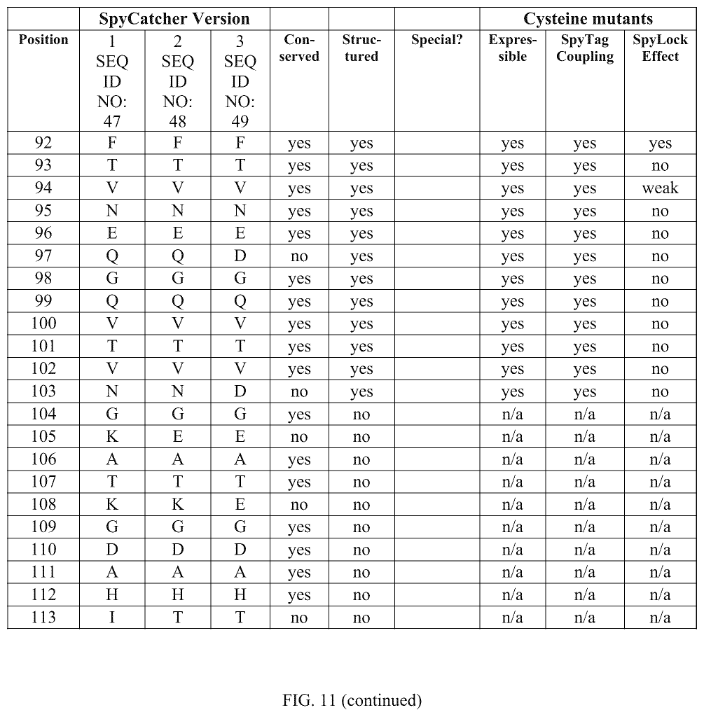

In the crystal structure of SpyCatcher, 4MLI, (Li, L., J. O. Fierer, T. A. Rapoport, and M. Howarth. “Structural Analysis and Optimization of the Covalent Association between SpyCatcher and a Peptide Tag.” J Mol Biol 426, no. 2 (2014): 309-17. Doi.org/10.1016/j.jmb.2013.10.021.), amino acid residues 22 to 103 could be resolved and were thus chosen for further screening and each amino acid was replaced by cysteine separately. Two negative controls, the isopeptide bond forming Lys31 and the catalytic Glu77, were included, but the already tested or known residues (see above) were excluded. This yielded a total of 75 constructs, that were expressed and purified (FIG. 11).

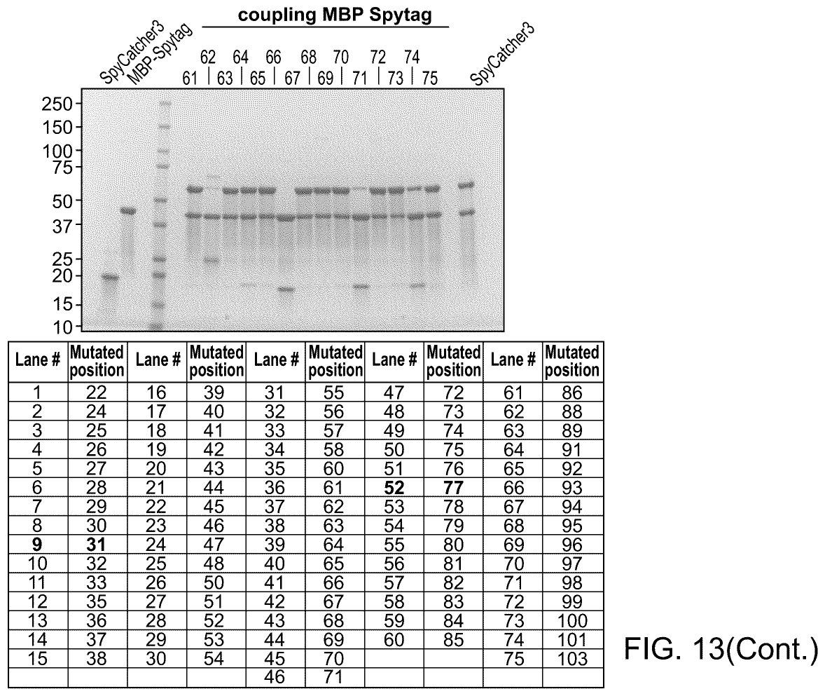

Next, coupling reactions of the unmodified Cys Catchers were performed to evaluate their coupling capability in an unlabeled state (FIG. 13). Essentially all Catchers, except for the controls, could still couple. However, mutations Leu39Cys and Pro81Cys had strongly reduced coupling speeds and were excluded from further analysis. Tyr73Cys had an unmeasurably low concentration of purified protein and thus was also excluded from follow-up, as were the two controls. The remaining Cys-Catchers were modified with Ellman's reagent (5,5′-dithiobis-(2-nitrobenzoic acid) to see if coupling could be inhibited. As expected from the fact that all 8 amino acid positions responsible for SpyLock behavior are conserved between SpyCatcher001, 002, and 003, S59C mutants of SpyCatcher001 and 002 work equivalently.

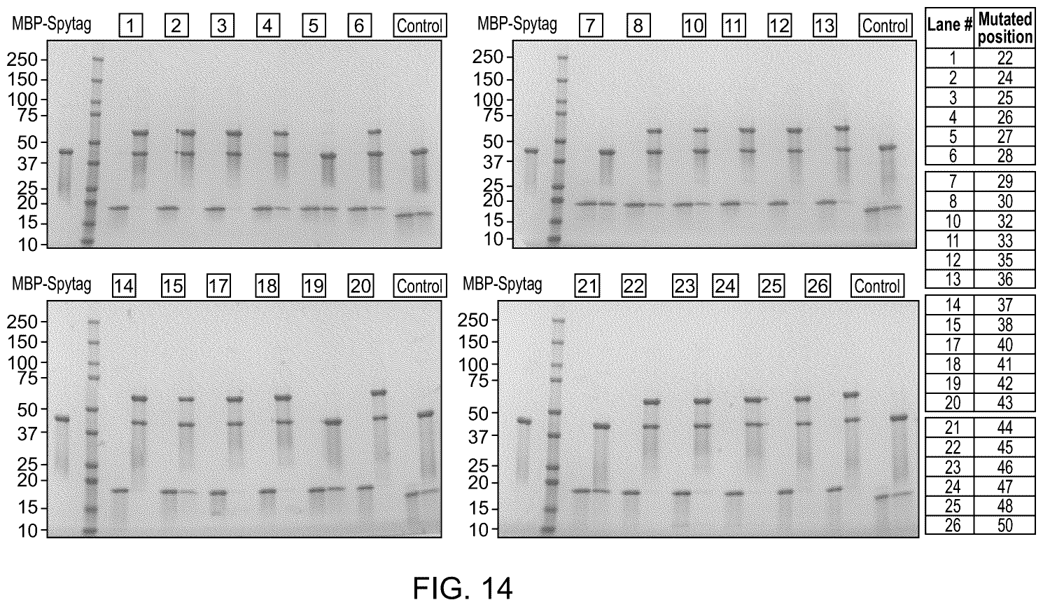

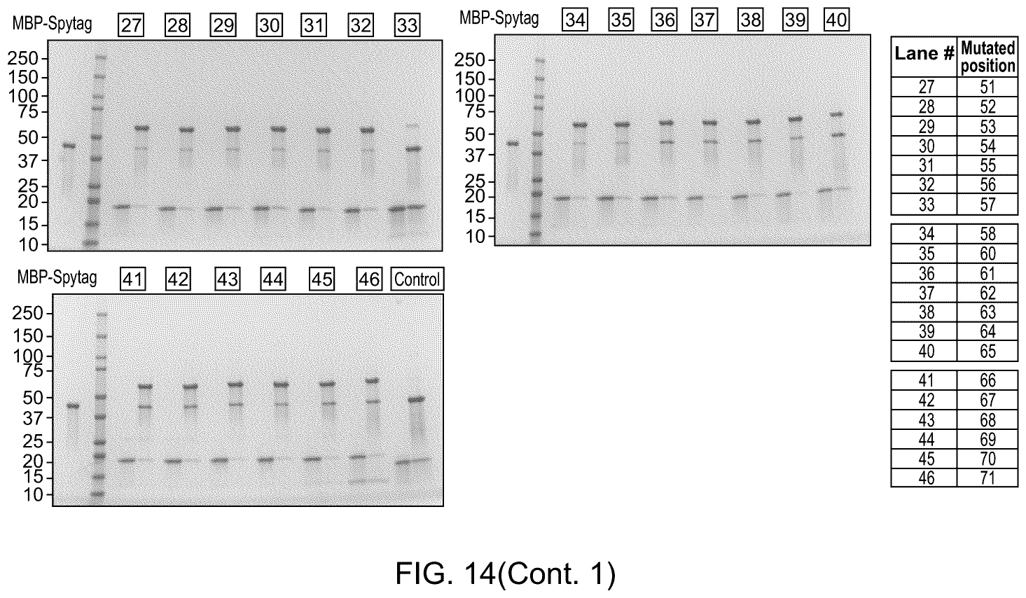

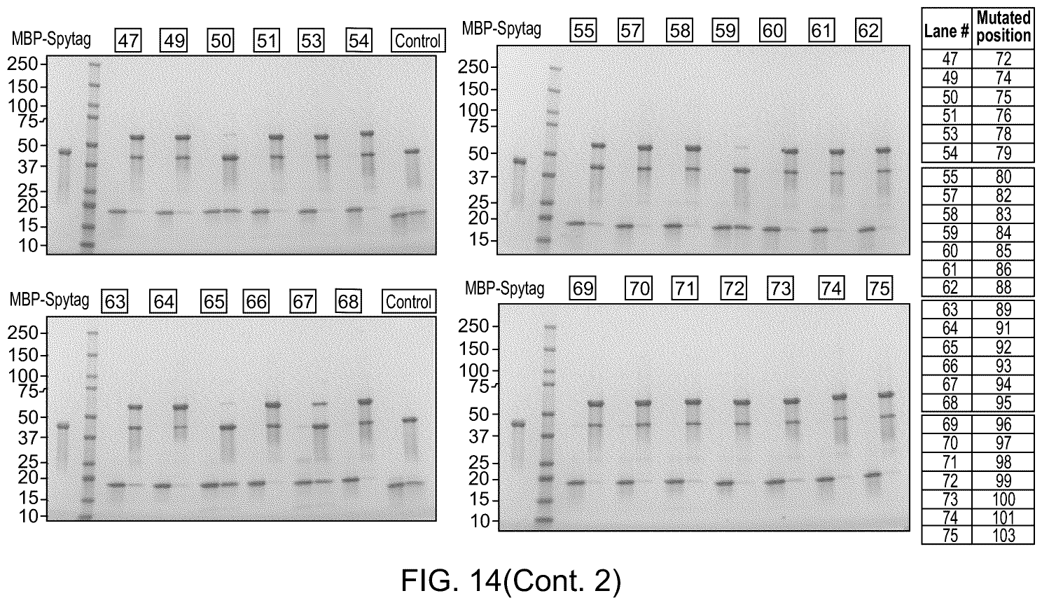

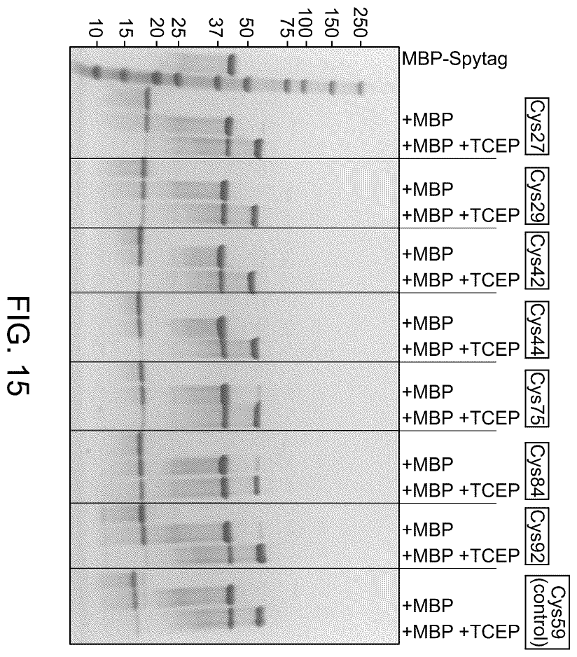

Indeed, several of the Ellman's reagent modified Cys-SpyCatchers could not couple to MBP-SpyTag and were thus candidates for new SpyLocks (FIG. 14). Identified candidates with strong regular coupling and strong SpyLock effects were: Iso27→Cys; Phe29→Cys; Ala42→Cys; Met44→Cys; Phe75→Cys; and Phe92→Cys. Additionally, Trp57→Cys and Tyr84→Cys showed a SpyLock effect, but their regular SpyTag coupling capability was also lowered. Furthermore, Val94→Cys and, as described above, Ala87→Cys showed a weak SpyLock effect.

To confirm the SpyLock effect of the final candidates, DTNB-modified Cys-Catchers were incubated with 10 mM TCEP to fully reduce the cysteine (i.e., “unlock” the SpyLock) and MBP-SpyTag (FIG. 15). Coupling activity could be restored for all tested SpyCatcher mutants. Tyr84→Cys was included in this test as well and exhibited reduced coupling speed after unlocking.

We claim:

1. A SpyCatcher analog comprising:
a) a SpyCatcher analog comprising any one of SEQ ID NOs: 1-4 or 7-46 or a mutated SpyCatcher analog having at least 60% sequence identity to said SpyCatcher analog, with the proviso that said mutated SpyCatcher analog having 60% sequence identity to said SpyCatcher analog catalyzes protein ligation and the cysteine residue introduced into said SpyCatcher analog is not substituted in said mutated SpyCatcher analog;
b) a SpyCatcher analog comprising one or more of the mutations in the SpyCatcher protein of SEQ ID NO: 47, said one or more mutations being selected from the group consisting of I27C, F29C, A42C, M44C, W57C, S59C, F75C, Y84C, A87C, F92C, and V94C, or a mutated SpyCatcher analog having at least 60% sequence identity to said SpyCatcher analog, with the proviso that said SpyCatcher analog or said mutated SpyCatcher analog catalyzes protein ligation and the cysteine residue(s) introduced into said SpyCatcher analog or said mutated SpyCatcher analog is/are not substituted;
c) a SpyCatcher analog comprising one or more of the mutations in the SpyCatcher protein of SEQ ID NO: 48, said one or more mutations being selected from the group consisting of I27C, F29C, A42C, M44C, W57C, S59C, F75C, Y84C, A87C, F92C, and V94C, or a mutated SpyCatcher analog having at least 60% sequence identity to said SpyCatcher analog, with the proviso that said SpyCatcher analog or said mutated SpyCatcher analog catalyzes protein ligation and the cysteine residue(s) introduced into said SpyCatcher analog or said mutated SpyCatcher analog is/are not substituted in said SpyCatcher analog or said polypeptide;
d) a SpyCatcher analog comprising one or more of the mutations in the SpyCatcher protein of SEQ ID NO: 49, said one or more mutations being selected from the group consisting of I27C, F29C, A42C, M44C, W57C, S59C, F75C, Y84C, A87C, F92C, and V94C, or a mutated SpyCatcher analog having at least 60% sequence identity to said SpyCatcher analog, with the proviso that said SpyCatcher analog or said mutated SpyCatcher analog catalyzes protein ligation and the cysteine residue(s) introduced into said SpyCatcher analog or said mutated SpyCatcher analog is/are not substituted in said SpyCatcher analog or said polypeptide;
e) a SpyCatcher analog comprising one or more of the mutations in the SpyCatcher protein of SEQ ID NO: 50, said one or more mutations being selected from the group consisting of I6C, F8C, A21C, M23C, W36C, S38C, F54C, Y63C, A66C, F71C, and V73C, or a mutated SpyCatcher analog having at least 60% sequence identity to said SpyCatcher analog, with the proviso that said SpyCatcher analog or said mutated SpyCatcher analog catalyzes protein ligation and the cysteine residue(s) introduced into said SpyCatcher analog or said mutated SpyCatcher analog is/are not substituted in said SpyCatcher analog or said polypeptide;
f) a SpyCatcher analog comprising one or more of the mutations in the SpyCatcher protein of SEQ ID NO: 47, said one or more mutations being selected from the group consisting of I27C, F29C, A42C, M44C, W57C, S59C, F75C, Y84C, A87C, F92C, and V94, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, wherein Lys31 and Glu77 are not substituted in said SpyCatcher analog or mutated SpyCatcher analog and the mutations introduced to form said SpyCatcher analog are not changed in the mutated SpyCatcher analog and the one or more mutations that are introduced to form the SpyCatcher analog of SEQ ID NO: 47 are: a) unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids; or b) cysteine residues in combination with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids;
wherein a SpyCatcher analog or mutated SpyCatcher analog containing one or more caged unnatural amino acids has the ability to catalyze protein ligation after uncaging of said one or more caged unnatural amino acids;
g) a SpyCatcher analog comprising one or more of the mutations in the SpyCatcher protein of SEQ ID NO: 48, said one or more mutations being selected from the group consisting of 127C, F29C, A42C, M44C, W57C, S59C, F75C, Y84C, A87C, F92C, and V94C, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, wherein Lys31 and Glu77 are not substituted in said SpyCatcher analog or mutated SpyCatcher analog and the mutations introduced to form said SpyCatcher analog are not changed in the mutated SpyCatcher analog and the one or more mutations that are introduced to form the SpyCatcher analog of SEQ ID NO: 48 are: a) unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids, preferably unnatural amino acids selected from photocaged cysteine, photocaged lysine, photocaged tyrosine, chemically caged lysines, or combinations of these unnatural amino acids; or b) cysteine residues in combination with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids, preferably unnatural amino acids selected from photocaged cysteine, photocaged lysine, photocaged tyrosine, chemically caged lysines, or combinations of these unnatural amino acids;
wherein a SpyCatcher analog or mutated SpyCatcher analog containing one or more caged unnatural amino acids has the ability to catalyze protein ligation after uncaging of said one or more caged unnatural amino acids;
h) a SpyCatcher analog comprising one or more of the mutations in the SpyCatcher protein of SEQ ID NO: 49, said one or more mutations being selected from the group consisting of T27C, F29C, A42C, M44C, W57C, S59C, F75C, Y84C, A87C, F92C, and V94C, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, wherein Lys31 and Glu77 are not substituted in said SpyCatcher analog or mutated SpyCatcher analog and the mutations introduced to form said SpyCatcher analog are not changed in the mutated SpyCatcher analog and the one or more mutations that are introduced to form the SpyCatcher analog of SEQ ID NO: 49 are: a) unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids, preferably unnatural amino acids selected from photocaged cysteine, photocaged lysine, photocaged tyrosine, chemically caged lysines, or combinations of these unnatural amino acids; or b) cysteine residues in combination with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids, preferably unnatural amino acids selected from photocaged cysteine, photocaged lysine, photocaged tyrosine, chemically caged lysines, or combinations of these unnatural amino acids;
wherein a SpyCatcher analog or mutated SpyCatcher analog containing one or more caged unnatural amino acids has the ability to catalyze protein ligation after uncaging of said one or more caged unnatural amino acids;
i) a SpyCatcher analog comprising one or more of the mutations in the SpyCatcher protein of SEQ ID NO: 50, said one or more mutations being selected from the group consisting of I6C, F8C, A21C, M23C, W36C, S38C, F54C, Y63C, A66C, F71C, and V73C, or a mutated SpyCatcher analog having at least 60%, 65%, 70%, 75%, 80%, 85%, 90%, 91%, 92%, 93%, 94%, 95%, 96%, 97%, 98%, or 99% identity sequence identity to said SpyCatcher analog, wherein the isopeptide forming Lys and catalytic Glu are not substituted in said SpyCatcher analog or mutated SpyCatcher analog and the mutations introduced to form said SpyCatcher analog are not changed in the mutated SpyCatcher analog and the one or more mutations that are introduced to form the SpyCatcher analog of SEQ ID NO: 50 are: a) unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids, preferably unnatural amino acids selected from photocaged cysteine, photocaged lysine, photocaged tyrosine, chemically caged lysines, or combinations of these unnatural amino acids; or b) cysteine residues in combination with unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids, preferably unnatural amino acids selected from photocaged cysteine, photocaged lysine, photocaged tyrosine, chemically caged lysines, or combinations of these unnatural amino acids;
wherein a SpyCatcher analog or mutated SpyCatcher analog containing one or more caged unnatural amino acids has the ability to catalyze protein ligation after uncaging of said one or more caged unnatural amino acids; or
j) a SpyCatcher, SpyCatcher002, SpyCatcher003, or SpyCatcher short analog in which the reactive lysine is “locked” with a photocaged lysine, such as (S)-2-Amino-6-((((7-hydroxy-2-oxo-2H-chromen-4-yl)methoxy)carbonyl)amino)hexanoic acid or a chemically caged lysine such as N6-(((4-azidobenzyl)oxy)carbonyl)-L-lysine).

2. A fusion protein comprising at least one SpyCatcher analog according to claim 1 and at least one non-mutated protein selected from the group consisting of SEQ ID NO: 47, 48, 49, 50, 51, 56, or 58 and/or at least one a mutated protein having at least 60% sequence identity to SEQ ID NO: 47, 48, 49, 50, 51, 56, or 58,
with the proviso that:
a) said mutated protein having 60% sequence identity to SEQ ID NO: 47, 48, 49, 50, 51, 56, or 58 catalyzes protein ligation.

3. The fusion protein according to claim 2, wherein the binding motifs are joined to one another via a linker.

4. The fusion protein according to claim 3, wherein the linker is a heterobifunctional chemical linker or is a peptide linker.

5. The fusion protein according to claim 2, wherein the linker is a peptide linker, such as -(GGGS)n-, where n is 1, 2, 3, 4, or 5, (Gly)n, where n is an integer between 2 and 50, Gly-Ser, Gly-Ser-Gly-Ser, Ala-Ser, Gly-Gly-Gly-Ser, Gly₄-Ser, (Gly₄-Ser)₂, (Gly₄-Ser)₃, (Gly₄-Ser)₄, (Gly₄-Ser)₂-Gly-Ala-Gly-Ser-Gly₄-Ser, Gly-(Gly₄-Ser)₂, Gly₄-Ser-Gly, Gly-Ser-Gly₂, Gly-Ser-Gly₂-Ser, ENLYFQG (SEQ ID NO: 61), GSENLYFQGSG (SEQ ID NO: 62), Ala-(Glu-Ala-Ala-Ala-Lys)ₙ-Ala (n=1-5) or GAP (Gly Ala Pro) sequence and the peptide linker may be the same or different between elements of the fusion protein.

6. An antigen binding protein comprising:
one or more first antigen binding fragments comprising a binding motif comprising a SpyTag analog or any one of SEQ ID NOs: 52-55 and, optionally, one or more second antigen binding fragments comprising a binding motif comprising a SpyTag analog or any one of SEQ ID NOs: 52-55 and a fusion protein comprising a SpyCatcher analog according to claim 2,
wherein the binding motifs are covalently conjugated to the fusion protein via protein ligation, provided that:
a) if one of the elements of the fusion protein is SEQ ID NO: 51, then one of the binding motifs of the antigen binding fragments is SEQ ID NO: 55 or a sequence having at least 60% identity to SEQ ID NO: 55;
b) if one of the elements of the fusion protein is SEQ ID NO: 56, then one of the binding motifs of the antigen binding fragments is SEQ ID NO: 57 or a sequence having at least 60% identity to SEQ ID NO: 57; and/or
c) if one of the elements of the fusion protein is SEQ ID NO: 58, then one of the binding motifs of the antigen binding fragments is SEQ ID NO: 59 or a sequence having at least 60% identity to SEQ ID NO: 59.

7. The antigen binding protein according to claim 6, wherein the antigen binding protein is monospecific and dimeric, monospecific and multimeric, bispecific, bispecific and dimeric, or bispecific and multimeric, or multispecific and multimeric.

8. The antigen binding protein according to claim 6, wherein the binding motifs are located at a C terminus, an N-terminus or embedded within an amino acid sequence of the antigen binding fragments.

9. The antigen binding protein according to claim 6, wherein the fusion protein or polypeptide further comprises a detectable label.

10. A solid support comprising a SpyCatcher analog according to claim 1 or a fusion protein comprising said SpyCatcher analog.

11. The solid support according to claim 10, wherein the SpyCatcher analog or the SpyCatcher analog of the fusion protein is locked with a disulfide forming reagent, such as Npys-Cys-StrepTag2 or other Npys-Cys-peptides, Ellmann's reagent (5,5′-dithio-bis-(2-nitrobenzoic acid), DTNB) or HPDP-biotin (N-[6-(biotinamido)hexyl]-3′-(2′-pyridyldithio)propionamide) or other disulfide forming reagents or other chemical reagents which can be selectively removed, e.g., by photo- or chemical uncaging, or by introduction of a caged unnatural amino acid that can be uncaged photochemically or chemically, for example, a caged aspartic acid, a caged serine, a caged glycine, a caged tyrosine, a caged cysteine, a caged lysine, a caged phosphoserine, or a caged phosphotyrosine.

12. A SpyLock protein comprising a SpyCatcher analog according to claim 1, said SpyLock protein being locked with: a) a reagent that forms a disulfide bond (a disulfide forming reagent) with the cysteine that has been introduced into said SpyCatcher analog; b) by formation of intra-chain disulfide bonds between cysteine residues introduced into said SpyCatcher analog; or c) with an unnatural amino acid selected from photocaged cysteine, photocaged lysine, photocaged tyrosine, chemically caged lysine, or combinations of these unnatural amino acids.

13. The SpyLock protein according to claim 12, wherein said SpyLock protein has been locked with an unnatural amino acid, or by formation of a disulfide bond by reaction of one or more introduced cysteines with Npys-Cys-StrepTag2, Npys-Cys-His6, Npys-Cys-Flag, Npys-Cys-myc, Ellmann's reagent (5,5′-dithio-bis-(2-nitrobenzoic acid), DTNB) or HPDP-biotin (N-[6-(biotinamido)hexyl]-3′-(2′-pyridyldithio)propionamide) or other disulfide forming reagents.

14. A fusion protein according to claim 2, said fusion protein having been locked with: a) a reagent that forms a disulfide bond with the cysteine that has been introduced into said SpyCatcher analog; b) by formation of intra-chain disulfide bonds between cysteine residues introduced into said SpyCatcher analog; or c) with one or more unnatural amino acid selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids.

15. The fusion protein according to claim 14, wherein said fusion protein has been locked with an unnatural amino acid, preferably a photocaged cysteine, photocaged lysine, photocaged tyrosine or chemically caged lysine, or by formation of a disulfide bond by reaction of one or more introduced cysteines with Npys-Cys-StrepTag2, Npys-Cys-His6, Npys-Cys-Flag, Npys-Cys-myc, Npys-Cys-peptide, Ellmann's reagent (5,5′-dithio-bis-(2-nitrobenzoic acid), DTNB) or HPDP-biotin (N-[6-(biotinamido)hexyl]-3′-(2′-pyridyldithio)propionamide) or other disulfide forming reagents.

16. A polynucleotide sequence encoding a SpyCatcher analog according to claim 1 or a fusion protein comprising said SpyCatcher analog, a nucleic acid construct comprising said polynucleotide sequence or a vector comprising said polynucleotide.

17. A host cell comprising a polynucleotide according to claim 16, a nucleic acid construct comprising said polynucleotide, or a vector comprising said polynucleotide or said nucleic acid construct.

18. A method for identifying lockable SpyCatcher, SnoopCatcher, DogCatcher, or SilkCatcher protein comprising:
a) introducing one or more amino acid mutation(s) into a protein comprising any one of SEQ ID NO: 47-51, 56, or 58 or a protein having at least 60% sequence identity to any one of SEQ ID NO: 47-51, 56, or 58 and catalyzing protein ligation, said one or more amino acid mutation(s) comprising:
i) the introduction of one or more cysteine residue as said one or more amino acid mutations; or
ii) introduction of one or more unnatural amino acid selected from a photocaged cysteine, photocaged lysine, photocaged tyrosine, chemically caged lysines, or combinations of these unnatural amino acids, wherein the introduction of the one or more unnatural amino acid forms a locked mutated protein; or
iii) the introduction of one or more cysteine residues and one or more unnatural amino acids selected from photocaged cysteine, photocaged lysine, chemically caged lysine, photocaged aspartic acid, chemically caged aspartic acid, photocaged serine, chemically caged serine, photocaged glycine, chemically caged glycine, photocaged phosphoserine, chemically caged phosphoserine, photocaged phosphotyrosine, chemically caged phosphotyrosine, photocaged tyrosine, chemically caged tyrosine, or combinations of these unnatural amino acids, preferably unnatural amino acids selected from photocaged cysteine, photocaged lysine, photocaged tyrosine, chemically caged lysines, or combinations of these unnatural amino acids, preferably unnatural amino acids selected from photocaged cysteine, photocaged lysine, photocaged tyrosine, chemically caged lysines, or combinations of these unnatural amino acids;
b) contacting a protein mutated to contain one or more cysteine residue with a locking agent, such as Ellmann's reagent (5,5′-dithio-bis-(2-nitrobenzoic acid), DTNB) or HPDP-biotin (N-[6-(biotinamido)hexyl]-3′-(2′-pyridyldithio)propionamide), that forms a disulfide bond with the cysteine introduced into said mutated protein and forming a disulfide bond between said cysteine and said locking agent to form a potentially locked mutated protein or forming intra-chain disulfide bonds between two cysteine residues that have been introduced into said mutated protein to form a potentially locked mutated protein; and
c) contacting said potentially locked mutated protein with a substrate comprising a binding motif specific for said potentially locked mutated protein and determining if said potentially locked mutated protein has catalyzed peptide bond formation between said potentially locked mutated protein and said substrate comprising a binding motif specific for said locked mutated protein, wherein the lack of a peptide bond between said potentially locked mutated protein and said substrate comprising a binding motif specific for said potentially locked mutated protein identifies the potentially locked mutated protein as a lockable protein, while the formation of a peptide bond between said substrate comprising a binding motif specific for said potentially locked mutated protein and said potentially locked mutated protein identifies the mutation in said lockable protein as unable to lock the protein.

19. The method according to claim 18, wherein:
a) the protein into which one or more amino acid mutation is introduced is selected from the group consisting of SpyCatcher, SpyCatcher short, SpyCatcher002, SpyCatcher003, SnoopCatcher, DogCatcher, SilkCatcher; and
b) the substrate is selected from the group consisting of SpyTag, SpyTag002, SpyTag003, SnoopTag, DogTag, and SilkTag or analogs thereof that are, optionally, labeled.

20. A lockable analog of SpyCatcher, SpyCatcher002, SpyCatcher003, SnoopCatcher, DogCatcher, or SilkCatcher produced by the method of claim 18.


SEQUENCE LISTING (62 sequences)

SEQ ID NO: 1 - protein, 113 aa, synthetic construct
VDTLSGLSSEQGQSGDMTIEEDSATHIKFSKRDEDGKELAGATMELRDSSGKTISTWICDGQVKDFYLYPGKYTFVETAAPDGYEVATAITFTVNEQGQVTVNGKATKGDAHI

SEQ ID NO: 2 - protein, 113 aa, synthetic construct
VTTLSGLSGEQGPSGDMTTEEDSATHIKFSKRDEDGRELAGATMELRDSSGKTISTWICDGHVKDFYLYPGKYTFVETAAPDGYEVATAITFTVNEQGQVTVNGEATKGDAHT

SEQ ID NO: 3 - protein, 113 aa, synthetic construct
VTTLSGLSGEQGPSGDMTTEEDSATHIKFSKRDEDGRELAGATMELRDSSGKTISTWICDGHVKDFYLYPGKYTFVETAAPDGYEVATPIEFTVNEDGQVTVDGEATEGDAHT

SEQ ID NO: 4 - protein, 83 aa, synthetic construct
DSATHIKFSKRDEDGKELAGATMELRDSSGKTISTWICDGQVKDFYLYPGKYTFVETAAPDGYEVATAITFTVNEQGQVTVNG

SEQ ID NO: 5 - protein, 242 aa, synthetic construct
VTTLSGLSGEQGPSGDMTTEEDSATHIKFSKRDEDGRELAGATMELRDSSGKTISTWICDGHVKDFYLYPGKYTFVETAAPDGYEVATPIEFTVNEDGQVTVDGEATEGDAHTGSSGSGGGGSGGGGSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 6 - protein, 242 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGSGGGGSGGGGSGVTTLSGLSGEQGPSGDMTTEEDSATHIKFSKRDEDGRELAGATMELRDSSGKTISTWICDGHVKDFYLYPGKYTFVETAAPDGYEVATPIEFTVNEDGQVTVDGEATEGDAHT

SEQ ID NO: 7 - protein, 113 aa, synthetic construct
VDTLSGLSSEQGQSGDMTIEEDSATHCKFSKRDEDGKELAGATMELRDSSGKTISTWISDGQVKDFYLYPGKYTFVETAAPDGYEVATAITFTVNEQGQVTVNGKATKGDAHI

SEQ ID NO: 8 - protein, 113 aa, synthetic construct
VDTLSGLSSEQGQSGDMTIEEDSATHIKCSKRDEDGKELAGATMELRDSSGKTISTWISDGQVKDFYLYPGKYTFVETAAPDGYEVATAITFTVNEQGQVTVNGKATKGDAHI

SEQ ID NO: 9 - protein, 113 aa, synthetic construct
VDTLSGLSSEQGQSGDMTIEEDSATHIKFSKRDEDGKELAGCTMELRDSSGKTISTWISDGQVKDFYLYPGKYTFVETAAPDGYEVATAITFTVNEQGQVTVNGKATKGDAHI

SEQ ID NO: 10 - protein, 113 aa, synthetic construct
VDTLSGLSSEQGQSGDMTIEEDSATHIKFSKRDEDGKELAGATCELRDSSGKTISTWISDGQVKDFYLYPGKYTFVETAAPDGYEVATAITFTVNEQGQVTVNGKATKGDAHI

SEQ ID NO: 11 - protein, 113 aa, synthetic construct
VDTLSGLSSEQGQSGDMTIEEDSATHIKFSKRDEDGKELAGATMELRDSSGKTISTCISDGQVKDFYLYPGKYTFVETAAPDGYEVATAITFTVNEQGQVTVNGKATKGDAHI

SEQ ID NO: 12 - protein, 113 aa, synthetic construct
VDTLSGLSSEQGQSGDMTIEEDSATHIKFSKRDEDGKELAGATMELRDSSGKTISTWISDGQVKDFYLYPGKYTCVETAAPDGYEVATAITFTVNEQGQVTVNGKATKGDAHI

SEQ ID NO: 13 - protein, 113 aa, synthetic construct
VDTLSGLSSEQGQSGDMTIEEDSATHIKFSKRDEDGKELAGATMELRDSSGKTISTWISDGQVKDFYLYPGKYTFVETAAPDGCEVATAITFTVNEQGQVTVNGKATKGDAHI

SEQ ID NO: 14 - protein, 113 aa, synthetic construct
VDTLSGLSSEQGQSGDMTIEEDSATHIKFSKRDEDGKELAGATMELRDSSGKTISTWISDGQVKDFYLYPGKYTFVETAAPDGYEVCTAITFTVNEQGQVTVNGKATKGDAHI

SEQ ID NO: 15 - protein, 113 aa, synthetic construct
VDTLSGLSSEQGQSGDMTIEEDSATHIKFSKRDEDGKELAGATMELRDSSGKTISTWISDGQVKDFYLYPGKYTFVETAAPDGYEVATAITCTVNEQGQVTVNGKATKGDAHI

SEQ ID NO: 16 - protein, 113 aa, synthetic construct
VDTLSGLSSEQGQSGDMTIEEDSATHIKFSKRDEDGKELAGATMELRDSSGKTISTWISDGQVKDFYLYPGKYTFVETAAPDGYEVATAITFTCNEQGQVTVNGKATKGDAHI

SEQ ID NO: 17 - protein, 113 aa, synthetic construct
VTTLSGLSGEQGPSGDMTTEEDSATHCKFSKRDEDGRELAGATMELRDSSGKTISTWISDGHVKDFYLYPGKYTFVETAAPDGYEVATAITFTVNEQGQVTVNGEATKGDAHT

SEQ ID NO: 18 - protein, 113 aa, synthetic construct
VTTLSGLSGEQGPSGDMTTEEDSATHIKCSKRDEDGRELAGATMELRDSSGKTISTWISDGHVKDFYLYPGKYTFVETAAPDGYEVATAITFTVNEQGQVTVNGEATKGDAHT

SEQ ID NO: 19 - protein, 113 aa, synthetic construct
VTTLSGLSGEQGPSGDMTTEEDSATHIKFSKRDEDGRELAGCTMELRDSSGKTISTWISDGHVKDFYLYPGKYTFVETAAPDGYEVATAITFTVNEQGQVTVNGEATKGDAHT

SEQ ID NO: 20 - protein, 113 aa, synthetic construct
VTTLSGLSGEQGPSGDMTTEEDSATHIKFSKRDEDGRELAGATCELRDSSGKTISTWISDGHVKDFYLYPGKYTFVETAAPDGYEVATAITFTVNEQGQVTVNGEATKGDAHT

SEQ ID NO: 21 - protein, 113 aa, synthetic construct
VTTLSGLSGEQGPSGDMTTEEDSATHIKFSKRDEDGRELAGATMELRDSSGKTISTCISDGHVKDFYLYPGKYTFVETAAPDGYEVATAITFTVNEQGQVTVNGEATKGDAHT

SEQ ID NO: 22 - protein, 113 aa, synthetic construct
VTTLSGLSGEQGPSGDMTTEEDSATHIKFSKRDEDGRELAGATMELRDSSGKTISTWISDGHVKDFYLYPGKYTCVETAAPDGYEVATAITFTVNEQGQVTVNGEATKGDAHT

SEQ ID NO: 23 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGCEVATAITFTVNEQGQVTVNGEATKGDAHT

SEQ ID NO: 24 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVCTAITFTVNEQGQVTVNGEATKGDAHT

SEQ ID NO: 25 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATAITCTVNEQGQVTVNGEATKGDAHT

SEQ ID NO: 26 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATAITFTCNEQGQVTVNGEATKGDAHT

SEQ ID NO: 27 - protein, 113 aa, synthetic construct
VTTLSGLSGEQGPSGDMTTEEDSATHCKFSKRDEDGRELAGATMELRDSSGKTISTWISDGHVKDFYLYPGKYTFVETAAPDGYEVATPIEFTVNEDGQVTVDGEATEGDAHT

SEQ ID NO: 28 - protein, 113 aa, synthetic construct
VTTLSGLSGEQGPSGDMTTEEDSATHIKCSKRDEDGRELAGATMELRDSSGKTISTWISDGHVKDFYLYPGKYTFVETAAPDGYEVATPIEFTVNEDGQVTVDGEATEGDAHT

SEQ ID NO: 29 - protein, 113 aa, synthetic construct
VTTLSGLSGEQGPSGDMTTEEDSATHIKFSKRDEDGRELAGCTMELRDSSGKTISTWISDGHVKDFYLYPGKYTFVETAAPDGYEVATPIEFTVNEDGQVTVDGEATEGDAHT

SEQ ID NO: 30 - protein, 113 aa, synthetic construct
VTTLSGLSGEQGPSGDMTTEEDSATHIKFSKRDEDGRELAGATCELRDSSGKTISTWISDGHVKDFYLYPGKYTFVETAAPDGYEVATPIEFTVNEDGQVTVDGEATEGDAHT

SEQ ID NO: 31 - protein, 113 aa, synthetic construct
VTTLSGLSGEQGPSGDMTTEEDSATHIKFSKRDEDGRELAGATMELRDSSGKTISTCISDGHVKDFYLYPGKYTFVETAAPDGYEVATPIEFTVNEDGQVTVDGEATEGDAHT

SEQ ID NO: 32 - protein, 113 aa, synthetic construct
VTTLSGLSGEQGPSGDMTTEEDSATHIKFSKRDEDGRELAGATMELRDSSGKTISTWISDGHVKDFYLYPGKYTCVETAAPDGYEVATPIEFTVNEDGQVTVDGEATEGDAHT

SEQ ID NO: 33 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGCEVATPIEFTVNEDGQVTVDGEATEGDAHT

SEQ ID NO: 34 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVCTPIEFTVNEDGQVTVDGEATEGDAHT

SEQ ID NO: 35 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIECTVNEDGQVTVDGEATEGDAHT

SEQ ID NO: 36 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTCNEDGQVTVDGEATEGDAHT

SEQ ID NO: 37 - protein, 83 aa, synthetic construct
DSATHCKFSKRDEDGKELAGATMELRDSSGKTISTWISDGQVKDFYLYPGKYTFVETAAPDGYEVATAITFTVNEQGQVTVNG

SEQ ID NO: 38 - protein, 83 aa, synthetic construct
DSATHIKCSKRDEDGKELAGATMELRDSSGKTISTWISDGQVKDFYLYPGKYTFVETAAPDGYEVATAITFTVNEQGQVTVNG

SEQ ID NO: 39 - protein, 83 aa, synthetic construct
DSATHIKFSKRDEDGKELAGCTMELRDSSGKTISTWISDGQVKDFYLYPGKYTFVETAAPDGYEVATAITFTVNEQGQVTVNG

SEQ ID NO: 40 - protein, 83 aa, synthetic construct
DSATHIKFSKRDEDGKELAGATCELRDSSGKTISTWISDGQVKDFYLYPGKYTFVETAAPDGYEVATAITFTVNEQGQVTVNG

SEQ ID NO: 41 - protein, 83 aa, synthetic construct
DSATHIKFSKRDEDGKELAGATMELRDSSGKTISTCISDGQVKDFYLYPGKYTFVETAAPDGYEVATAITFTVNEQGQVTVNG

SEQ ID NO: 42 - protein, 83 aa, synthetic construct
DSATHIKFSKRDEDGKELAGATMELRDSSGKTISTWISDGQVKDFYLYPGKYTCVETAAPDGYEVATAITFTVNEQGQVTVNG

SEQ ID NO: 43 - protein, 83 aa, synthetic construct
DSATHIKFSKRDEDGKELAGATMELRDSSGKTISTWISDGQVKDFYLYPGKYTFVETAAPDGCEVATAITFTVNEQGQVTVNG

SEQ ID NO: 44 - protein, 83 aa, synthetic construct
DSATHIKFSKRDEDGKELAGATMELRDSSGKTISTWISDGQVKDFYLYPGKYTFVETAAPDGYEVCTAITFTVNEQGQVTVNG

SEQ ID NO: 45 - protein, 83 aa, synthetic construct
DSATHIKFSKRDEDGKELAGATMELRDSSGKTISTWISDGQVKDFYLYPGKYTFVETAAPDGYEVATAITCTVNEQGQVTVNG

SEQ ID NO: 46 - protein, 83 aa, synthetic construct
DSATHIKFSKRDEDGKELAGATMELRDSSGKTISTWISDGQVKDFYLYPGKYTFVETAAPDGYEVATAITFTCNEQGQVTVNG

SEQ ID NO: 47 - protein, 113 aa, synthetic construct
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 48 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 49 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 50 - protein, 83 aa, synthetic construct
DSATHIKFSKRDEDGKELAGATMELRDSSGKTISTWISDGQVKDFYLYPGKYTFVETAAPDGYEVATAITFTVNEQGQVTVNG

SEQ ID NO: 51 - protein, 112 aa, synthetic construct
KPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 52 - protein, 13 aa, synthetic construct
AHIVMVDAYKPTK

SEQ ID NO: 53 - protein, 14 aa, synthetic construct
VPTIVMVDAYKRYK

SEQ ID NO: 54 - protein, 16 aa, synthetic construct
RGVPHIVMVDAYKRYK

SEQ ID NO: 55 - protein, 12 aa, synthetic construct
KLGDIEFIKVNK

SEQ ID NO: 56 - protein, 104 aa, synthetic construct
KLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 57 - protein, 23 aa, synthetic construct
DIPATYEFTDGKHYITNEPIPPK

SEQ ID NO: 58 - protein, 79 aa, synthetic construct
SMTYTIELTKHDAATKAVLAGAVYELQDSTGKVIQTGLTTDSQGQLIVKNLRAGDYQFVETKAPLGYELNTTPVKFTLG

SEQ ID NO: 59 - protein, 20 aa, synthetic construct
GIKPEVAFQVSQDDVKQPVV

SEQ ID NO: 60 - protein, 5 aa, synthetic construct
  source 1..5: note = linker
GGGGS

SEQ ID NO: 61 - protein, 7 aa, synthetic construct
  source 1..7: note = linker
ENLYFQG

SEQ ID NO: 62 - protein, 11 aa, Tobacco etch virus
GSENLYFQGSG